(19) United States
(12) Patent Application Publication
(10) Pub. No.: US 2022/0267258 A1
(43) Pub. Date: Aug. 25, 2022

(54) ROBENIDINE ANALOGS AS POTENT ANTIMALARIALS AGAINST DRUG-RESISTANT PLASMODIUM FALCIPARUM

(71) Applicants: OREGON HEALTH & SCIENCE UNIVERSITY, Portland, OR (US); THE UNITED STATES GOVERNMENT AS REPRESENTED BY THE DEPARTMENT OF VETERANS, WASHINGTON, DC (US)

(72) Inventors: Alina Krollenbrock, mILWAUKIE, OR (US); Michael K. Riscoe, Portland, OR (US)

(21) Appl. No.: 17/592,366

(22) Filed: Feb. 3, 2022

Related U.S. Application Data
(60) Provisional application No. 63/145,354, filed on Feb. 3, 2021.

(51) Int. Cl. C07C 281/18; A61P 33/06

(52) U.S. Cl. CPC C07C 281/18; A61P 33/06

(57) ABSTRACT

Provided herein is a compound of Formula (I):

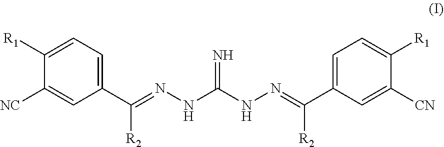

wherein:
R₁ is selected from the group of —F, —Cl, —Br, —I, C₁-C₆ haloalkyl, C₁-C₆ haloalkoxy, —NO₂, —C(H)═O, ═O, —CN, and COOR₃;
R₂ is selected from the group of H and C₁-C₃ alkyl; and
R₃ is selected from the group of H, C₁-C₆ alkyl, and benzyl;
   or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, along with pharmaceutical compositions and methods of using the compound in the treatment of malaria, particularly including drug-resistant malaria.

### STATEMENT OF GOVERNMENT SUPPORT

This invention was made with government support under R01 AI100569 and R01 Al141412 awarded by the National Institutes of Health and W81XWH-19-2-0031 awarded by the Department of Defense. The government has certain rights in the invention.

### BACKGROUND OF THE INVENTION

Robenidine is a veterinary drug used in the poultry industry to treat coccidiosis caused by parasites in the Eimeria genus. Though this compound and related aminoguanidines have recently been studied in other pathogens, the chemotype has not been systematically explored to optimize antimalarial activity despite the close genetic relationship between Eimeria and Plasmodium (both are members of the Apicomplexa phylum of unicellular, spore-forming parasites).

Malaria remains a devastating parasitic disease responsible for 229 million infections and 409,000 deaths in 2019.¹ Among the species of malaria parasites that can infect humans, Plasmodium falciparum is the most virulent and deadly. Pregnant women and children are the most vulnerable to mortality from malaria, and most cases and deaths occur in Sub-Saharan Africa, where P. falciparum is the dominant species. Widespread resistance to existing therapies for malaria is an increasingly significant concern for global health. Drug resistance continues to spread for frontline malaria therapies, and resistance has emerged in the last decade in Cambodia and neighboring countries for current ‘last line of defense’ drugs such as artemisinin.² The spread of artemisinin resistance outside of Southeast Asia seems inevitable and threatens to reverse the recent progress that has been made in decreasing malaria deaths worldwide, i.e., from the year 2000 to 2019 deaths have fallen from 736,000 to 409,000.

Development of new malaria drug classes that can evade existing resistance mechanisms is an urgent global need if elimination and eradication goals are to be achieved.³ One frequently proven successful approach to the development of novel therapeutics is to begin with a drug for a related parasite or pathogen and refine its structure using medicinal chemistry techniques.

Robenidine (FIG. 1a), formerly called Robenzidene, is a veterinary drug developed in the early 1970's to treat coccidiosis caused by parasites in the Eimeria genus⁴⁻¹⁰. Robenidine is frequently added to animal feed to prevent and treat coccidiosis in chickens,¹¹ other fowl,¹² and rabbits.¹³ Eimeria and Plasmodium, the parasite responsible for malaria infections, are both members of the Apicomplexan phylum, distinguished by the presence of the apical complex and frequent presence of the apicoplast organelle.¹⁴

Robenidine and other aminoguanidines have been evaluated for efficacy against several other protozoan parasites, including Toxoplasma gondii (in vitro IC₅₀ 0.03 μg/mL for robenidine),¹⁵⁻¹⁸ Leishmania donovani (in vitro IC₅₀ 18 μM for the analog CGP 40215A),¹⁹ Babesia microti (murine in vivo non-recrudescence dose 100 mg/kg/day for oral robenidine),^{20, 21} and the trypanosomes, T. brucei and T. cuzi (in vitro IC₅₀ 20 μM for the analog CGP 40215A).²²⁻²⁴ It has also been tested against microorganisms such as Lactobacillus,²⁵ E. coli,²⁶ Acanthamoeba polyphaga,²⁷ Goussia carpelli,²⁸ and several other gram positive and gram negative bacterial pathogens.^{29, 30}

Recently, work by Trott and McClusky³¹⁻³⁶ has further explored the aminoguanidine chemotype of robenidine, applying a medicinal chemistry approach to repurpose the drug for various bacterial pathogens. Their ongoing research has demonstrated that this chemical scaffold is amenable to synthetic modification and can successfully be refined for activity against pathogens other than Eimeria.

Despite significant genetic similarity between Eimeria and Plasmodium, there has been relatively little research into the efficacy of robenidine against malaria in its 50-year history. Robenidine was evaluated against the murine species P. berghei during its initial development in 1970 and found to have moderate activity in vivo.⁴ An immune analog of robenidine, CGP 40215A, was tested against the human pathogen P. falciparum in vitro with low micromolar IC₅₀ value.³⁷ To date, no medicinal chemistry efforts have focused on refining and optimizing the aminoguanidine scaffold for antimalarial activity.

Substituted benzylguanidine derivatives have been described for anti-bacterial, anti-protozoan, insecticidal, and other uses, including those described by Peng et al. in U.S. Pat. No. 9,663,458 and US patent publication no. 2020/0016099 and by Page et al. in U.S. Pat. Nos. 10,253,002, 10,370,341, 10,562,880, 10, 752,606, and 10,829,469. Others include those of Kulsa et al. in U.S. Pat. Nos. 3,795,692 and 3,897,563, Tomcufcik et al. in U.S. Pat. Nos. 3,901,944, 3,941,825, and 3,992,446, Militzer et al. in U.S. Pat. No. 3,950,539, Wang et al. in U.S. Pat. No. 4,310,541, and Addor et al. in U.S. Pat. No. 4,575,560.

There remains a need for improved antimalarial agents, particularly those effective against drug-resistant P. falciparum.

### SUMMARY OF THE INVENTION

One embodiment provided herein comprises a compound of Formula (I):

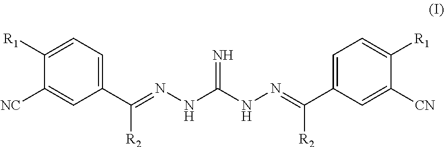

wherein:

R₁ is selected from the group of —F, —Cl, —Br, —I, C₁-C₆ haloalkyl, C₁-C₆ haloalkoxy, —NO₂, —C(H)═O, ═O, —CN, and COOR₃;

R₂ is selected from the group of H and C₁-C₃ alkyl; and

R₃ is selected from the group of H, C₁-C₆ alkyl, and benzyl;

or a pharmaceutically acceptable co-crystal, ester, solvate, hydrate, isomer (including optical isomers, racemates, or other mixtures thereof), tautomer, isotope, polymorph, or pharmaceutically acceptable prodrug thereof.

### BRIEF DESCRIPTION OF THE MANY VIEWS OF THE DRAWINGS

FIG. 1 represents an IC₅₀ curve of robenidine in D6 compared with chloroquine, wherein Y values represent fluorescence (reflecting parasite growth) relative to untreated controls.

FIG. 2 graphs Inhibition of P. falciparum Dd2 growth by AK3058 (green) and AK3072 (purple) at 48 hr (dotted curves), 72 hr (dashed curves), and 96 hr (solid curves) time points.

### DETAILED DESCRIPTION OF THE INVENTION

As described herein, a series of aminoguanidine robenidine analogs were prepared and tested in vitro against Plasmodium falciparum, including multi-drug-resistant strains. Selected compounds were further evaluated in vivo against murine Plasmodium yoelii in mice. Iterative structure activity relationship studies led to the discovery of Compound 1, an aminoguanidine with excellent activity against drug-resistant malaria in vitro and impressive in vivo efficacy with ED₅₀ value of 0.25 mg/kg/day in a standard 4-day test.

Another embodiment provides a compound of Formula (I):

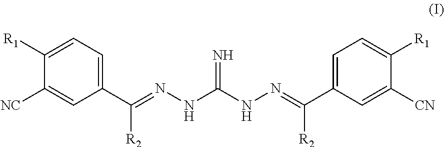

wherein:

R₁ is selected from the group of —F, —Cl, —Br, C₁-C₃ haloalkyl, and C₁-C₃ haloalkoxy;

R₂ is selected from the group of H and C₁-C₃ alkyl; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R1 is selected from the group of —F, —Cl, C₁-C₃ haloalkyl, and C₁-C₃ haloalkoxy;

R2 is selected from the group of H and C₁-C₃ alkyl; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R₁ is selected from the group of —F, —Cl, C₁-C₃ fluoroalkyl, and C₁-C₃ fluoroalkoxy;

R₂ is selected from the group of H and C₁-C₃ alkyl; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R₁ is selected from the group of —F, —Cl, C₁-C₂ fluoroalkyl, and C₁-C₂ fluoroalkoxy;

R₂ is selected from the group of H and C₁-C₃ alkyl; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R₁ is selected from the group of —F, —Cl, —CH₂F, CH F₂, —CF₃, —OCH₂F, —OCHF₂, and —OCF₃;

R₂ is selected from the group of H and C₁-C₃ alkyl; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A further embodiment provides a compound of Formula (I), wherein:

R₁ is selected from the group of —F, —Cl, —CH₂F, CHF₂, —CF₃, —OCH₂F, —OCHF₂, and —OCF₃;

R₂ is selected from the group of H and —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

An additional embodiment provides a compound of Formula (I), wherein:

R₁ is selected from the group of —F, —Cl, —CH₂F, CH F₂, —CF₃, —OCH₂F, —OCHF₂, and —OCF₃;

R₂ is H; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R₁ is selected from the group of —F, —Cl, —CH₂F, CH F₂, —CF₃, —OCH₂F, —OCHF₂, and —OCF₃;

R₂ is —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still further embodiment provides a compound of Formula (I), wherein:

R₁ is selected from the group of —F and —Cl;

R₂ is selected from the group of H and —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Another embodiment provides a compound of Formula (I), wherein:

R₁ is selected from the group of —F and —Cl;

R₂ is H; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still further embodiment provides a compound of Formula (I), wherein:

R₁ is selected from the group of —F and —Cl;

R₂ is —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still further embodiment provides a compound of Formula (I), wherein:

R₁ is —F;

R₂ is selected from the group of H and —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

A still further embodiment provides a compound of Formula (I), wherein:

R₁ is —Cl;

R₂ is selected from the group of H and —CH₃; and

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

It is understood that embodiments herein include those in which R₁ in each instance is the same and R₂ in each instance is the same, creating symmetrical molecules. For instance, when R₁ is F, it is F in the positions identified on both sides of the molecule. Similarly, when R₂ is H, it is H on both R₂ positions of the molecule. These symmetrical arrangements are indicated in compounds herein.

Also provided herein is a pharmaceutical composition comprising a pharmaceutically effective amount of a compound of any of the embodiments and specific compounds described herein encompassed by such embodiment or embodiments, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, and a pharmaceutically acceptable carrier.

Also provided herein are methods of treating human and veterinary diseases caused by parasites of the phylum Apicomplexa (also known as Apicomplexia).

Provided herein is a method of treatment of a malaria infection in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the malaria infection in the subject is caused by a Plasmodium species selected from the group of Plasmodium falciparum, Plasmodium vivax, Plasmodium ovale, Plasmodium malariae, and Plasmodium knowlesi. In some embodiments the malaria infection in the subject is caused by a species selected from Plasmodium falciparum and Plasmodium vivax. In some embodiments the malaria infection in the subject is caused by Plasmodium falciparum.

In some embodiments, the infecting species, such as a Plasmodium species, is resistant to one or more anti-malarial agents or combinations thereof, including those selected from the group of chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, artemisinin derivatives (including dihydroaremisinin (DHA), artesunate, artmether, arteether), artemisinin-based combination therapies (ACT), such as DHA-piperaquine and DHA-piperaquine mefloquine-artesunate. It is understood that reference to one or more of these antimalarial agents includes pharmaceutically acceptable salts thereof.

In some embodiments, the infecting agent is resistant to one antimalarial agent, which may be referred to as drug resistant malaria.

A multi-drug resistant malaria refers to a malarial infection that has proven to be resistant to treatment with two or more known agents for the treatment of malaria, or caused by an infective species known to be resistant to treatment with two or more of such, such as a multi-drug resistant Plasmodium species. In some embodiments, the multi-drug resistant species is a Plasmodium falciparum species.

Also provided is a method of treating coccidiosis in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the subject to be treated for coccidiosis is a human subject. In other embodiments, the subject is a veterinary subject. In some embodiments, the veterinary subject is poultry, such as a chicken. In other embodiments, the veterinary subject is a mammal subject, such as selected from the group of cattle, horses, dogs, cats, sheep, goats, pigs, and rabbits.

Another embodiment provides a method of treating coccidiosis in a poultry subject, the method comprising administering to a subject in need thereof:
a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a pharmaceutically effective amount of decoquinate, or a pharmaceutically acceptable salt thereof.

In some embodiments, the compound of Formula (I) is a administered to the poultry, such as chickens, in their feed at a concentration of from about 1 mg/kg to about 100 mg/kg.

In some embodiments, the decoquinate is also administered to the poultry, such as chickens, in their feed at a concentration of from about 1 mg/kg to about 100 mg/kg. In other embodiments, the decoquinate is present in the feed at a concentration of from about 30 mg/kg to about 50 mg/kg. In other embodiments, the decoquinate is present in the feed at a concentration of about 40 mg/kg.

In embodiments herein for treating veterinary subjects, it is understood that the active compound of Formula (I) or other pharmaceutical agents may be incorporated into animal feed at a desired concentration using techniques known in the art, including micronization or nanosization of the material, including the mechanical methods of milling, grinding, and cutting, as well as the use of supercritical fluids in supersaturation and precipitation techniques, such as Rapid Expansion of Supercritical Solutions (RESS), the Supercritical Anti-Solvent method (SAS), and Particles from Gas Saturated Solutions method (PGSS).

Also provided is a method of treating babesiosis (also known as a piroplasmosis) in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the subject of the piroplasmosis is infected by a Theileria species, such as T. equi (horses, donkeys, mules, and dogs), T. annae (dogs).

In other embodiments, the subject of the piroplasmosis is infected by a Babesia species, such as B. microti (human), B. duncani (human), Babesia canis (dogs), B. gibsoni (dogs), Babesia fells (cats), Babesia caballi (horses, donkeys, mules), Babesia ovis (sheep), Babesia motasi (sheep), Babesia odocoilei (deer and reindeer), B. orientalis (Buffalo).

In cattle, the Babesia infection in the subject may be from a species selected from the group of B. divergens, Babesia bigemina, Babesia bovis, B. beliceri, B. jakimovi, B. major, B. occultans, and B. ovata. In pigs, the Babesia infection in the subject may be from a species selected from the group of B. perroncitoi and B. trautmanni.

In small ruminants, such as sheep and goats, the Babesia infection in the subject may be from a species selected from the group of Babesia motasi, B. ovis, and B. crassa.

Another embodiment provides a method of treating babesiosis (also known as piroplasmosis) in a human subject, the method comprising administering to a subject in need thereof:
a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a pharmaceutically effective amount of atovaquone, or a pharmaceutically acceptable salt thereof; and
c) a pharmaceutically effective amount of azithromycin, or a pharmaceutically acceptable salt thereof.

In some embodiments, the method above comprises administering the atovaquone, or a pharmaceutically acceptable salt thereof, to the subject at a dosage of from about 500 mg to about 1,000 mg once or twice per day. In some embodiments, the atovaquone, or a pharmaceutically acceptable salt thereof, is administered to the subject at a dosage of about 750 mg once or twice per day.

Another embodiment provides a method of treating babesiosis (also known as piroplasmosis) in a human subject, the method comprising administering to a subject in need thereof:
d) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
e) a pharmaceutically effective amount of clindamycin, or a pharmaceutically acceptable salt thereof; and
f) a pharmaceutically effective amount of quinine, or a pharmaceutically acceptable salt thereof.

In some embodiments, the method above comprises administering the clindamycin, or a pharmaceutically acceptable salt thereof, to the subject at a dosage of from about 500 mg to about 750 mg once, twice, or three times per day. In some embodiments, the clindamycin, or a pharmaceutically acceptable salt thereof, is administered to the subject orally at a dosage of about 600 mg three times per day. In other embodiments, the clindamycin, or a pharmaceutically acceptable salt thereof, is administered intravenously to the subject at a dosage of from about 300 mg to about 600 mg three or four times per day.

In some embodiments, the method above comprises administering the quinine, or a pharmaceutically acceptable salt thereof, to the subject at a dosage of from about 500 mg to about 750 mg once, twice, or three times per day. In other embodiments, the quinine, or a pharmaceutically acceptable salt thereof, is administered orally to the subject at a dosage of about 650 mg three per day.

Another embodiment provides a method of treating babesiosis (also known as piroplasmosis) in a veterinary subject, the method comprising administering to a subject in need thereof:
a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a pharmaceutically effective amount of buparvaquone, or a pharmaceutically acceptable salt thereof; and
c) a pharmaceutically effective amount of azithromycin, or a pharmaceutically acceptable salt thereof.

In one embodiment, the veterinary subject in the method of treating babesiosis immediately above is a canine subject. In another embodiment, the veterinary subject in the method of treating babesiosis immediately above is a feline subject.

Also provided is a method of treating cryptosporidiosis (parasitic infection of a protozoan parasite of the genus Cryptosporidium) in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the treatment of cryptosporidiosis is in an immunocompromised human subject. In some embodiments, the immunocompromised human subject has an HIV infection or AIDS. In other embodiments, the immunocompromised human subject has an autoimmune disorder. In some embodiments, the cryptosporidiosis is intestinal cryptosporidiosis. In other embodiments, the cryptosporidiosis is respiratory cryptosporidiosis. In some embodiments, the cryptosporidiosis in the subject is caused by a Cryptosporidium parvum infection. In other embodiments, the cryptosporidiosis in the subject is caused by a C. meleagridis or C. fells infection.

Another embodiment provides a method of treating cryptosporidiosis in a human subject, the method comprising administering to a subject in need thereof:
c) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
d) a pharmaceutically effective amount of an agent selected from the group of nitazoxanide and azithromycin, or a pharmaceutically acceptable salt thereof.

Also provided is a method of treating cyclosporiasis in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the subject is human and the cyclosporiasis is caused by an infection of Cyclospora cayetanensis.

Another embodiment provides a method of treating cyclosporiasis in a human subject, the method comprising administering to a subject in need thereof:
a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a pharmaceutically effective amount of at least one agent selected from the group of trimethoprim and sulfamethoxazole, or a pharmaceutically acceptable salt thereof.

Another embodiment provides a method of treating isosporiasis (also known as cystoisosporiasis) in a human subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the isosporiasis is caused by an infection of Ispospora belli (now known as Cystoisospora belli).

Another embodiment provides a method of treating isosporiasis in a human subject, the method comprising administering to a subject in need thereof:
c) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
d) a pharmaceutically effective amount of at least one agent selected from the group of trimethoprim and sulfamethoxazole, or a pharmaceutically acceptable salt thereof.

In some embodiments, the treatment of isosporiasis is in an immunocompromised human subject. In some embodiments, the immunocompromised human subject has an HIV infection or AIDS. In other embodiments, the immunocompromised human subject has an autoimmune disorder.

Also provided is a method of treating toxoplasmosis in a subject, the method comprising administering to a subject in need thereof a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

In some embodiments, the toxoplasmosis is caused by an infection of Toxoplasma gondii.

Another embodiment provides a method of treating toxoplasmosis in a human subject, the method comprising administering to a subject in need thereof:
a) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a pharmaceutically effective amount of at least one agent selected from the group of trimethoprim and sulfamethoxazole, or a pharmaceutically acceptable salt thereof.

Another embodiment provides a method of treating toxoplasmosis in a human subject, the method comprising administering to a subject in need thereof:
c) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
d) a pharmaceutically effective amount of at least one agent selected from the group of pyrimethamine, sulfadiazine, clindamycin, spiramycin, minocycline, pyrimethamine, sulfadiazine, and leucovorin, or a pharmaceutically acceptable salt thereof.

In some embodiments of the two methods of treatment described immediately above that comprises administering pharmaceutically effective amounts of the compound of Formula (I) along with other pharmaceutical agents, the subject is a human experiencing an HIV infection. In other embodiments, the human subject is experiencing an immune disorder.

A further embodiment provides a method of treating latent toxoplasmosis in a human subject, the method comprising administering to a subject in need thereof:
e) a pharmaceutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
f) a pharmaceutically effective amount of at least one agent selected from the group of atovaquone and clindamycin, or a pharmaceutically acceptable salt thereof.

The term “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula (I), or a pharmaceutically acceptable salt or co-crystal thereof, is an amount sufficient to modulate a malarial infection (or other Apicomplexa infection described herein), and thereby treat a subject (e.g., a human) suffering from the infection, or to ameliorate or alleviate the existing symptoms of the infection. For example, a therapeutically or pharmaceutically effective amount may be an amount sufficient to decrease a symptom of a malarial infection (or other Apicomplexa infection described herein), as described herein.

In some embodiments, for human administration, each dosage unit contains from 0.1 mg to 1 g, 0.1 mg to 700 mg, or 0.1 mg to 100 mg of a compound of Formula (I), or a pharmaceutically acceptable salt or co-crystal thereof. In some embodiments, a therapeutically effective amount or a pharmaceutically effective amount of a compound of Formula (I), or a pharmaceutically acceptable salt thereof, comprises from about 0.1 mg to about 500 mg per dose, given once or twice daily. In some embodiments, the individual dose is selected from 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, 500 mg, 600 mg, 700 mg, 750 mg, 800 mg, 900 mg, and 1 g per dose.

The terms “subject,” “patient,” or “recipient” refer to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula (I), or a pharmaceutically acceptable salt or co-crystal thereof, as described herein. This includes a subject who may be determined to be at risk of or susceptible to such diseases or conditions, such that treatment would prevent the disease or condition from developing.

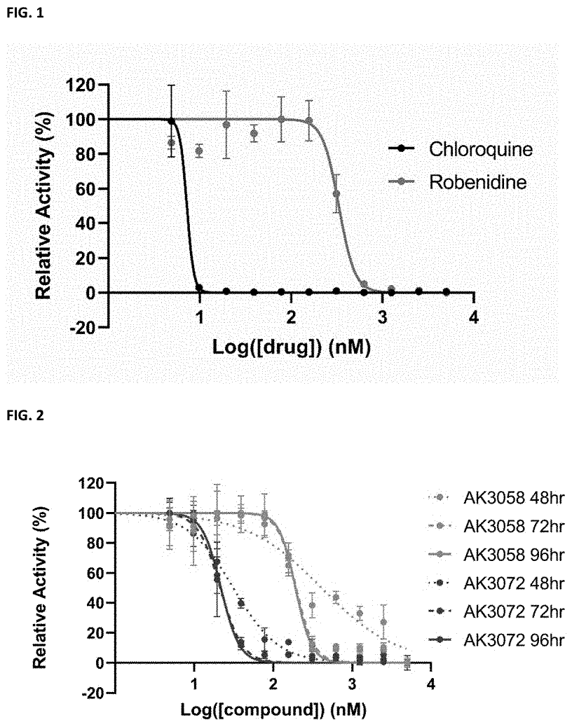

The current study represents the first attempt to evaluate structure-activity relationships (SAR) of the aminoguanidine chemotype against malaria parasites using in vitro assays and an in vivo murine model. A library of 38 structurally diverse aminoguanidines was created and compared for in vitro anti-plasmodial activity as well as mammalian cell cytotoxicity. Compounds with promising selective activity were further evaluated in vivo in a murine model of malaria. Multiple aminoguanidines from this library were found to have high potency in vitro which translated to robust efficacy in vivo. One compound, 1 (FIG. 1b), exhibits single-digit nanomolar IC₅₀ values against P. falciparum strains with an in vivo ED₅₀ value below 1 mg/kg/day vs. murine malaria.

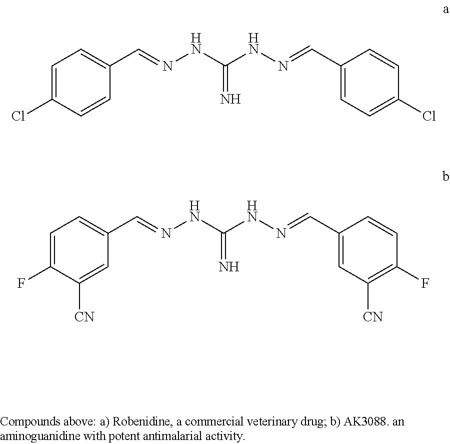 [SMILES: N=C(N/N=C/c1ccc(Cl)cc1)N/N=C/c1ccc(Cl)cc1 ; N#Cc1cc(/C=N/NC(=N)N/N=C/c2ccc(F)c(C#N)c2)ccc1F]

### Definitions

The term “alkyl” refers to a straight or branched hydrocarbon. For example, an alkyl group can have 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

The term “haloalkyl” refers to an alkyl group, as defined above, in which one or more hydrogen atoms of the alkyl group is replaced with a halogen atom. The alkyl portion of a haloalkyl group can have, for instance, 1 to 6 carbon atoms (i.e., C₁-C₆ haloalkyl), 1 to 4 carbon atoms (i.e., C₁-C₄ haloalkyl), 1 to 3 carbon atoms (i.e., C₁-C₃ haloalkyl), 1 to 1 carbon atoms (i.e., C₁-C₂ haloalkyl), or only one carbon atom C₁ haloalkyl or halomethyl). Non-limiting examples of suitable haloalkyl groups, which may also be referred to as halofluoro groups include, but are not limited to, trifluoromethyl (—CF₃), difluoromethyl (—CHF₂), fluoromethyl (—CFH₂), 2-fluoroethyl (—CH₂CH₂F), 2-fluoropropyl (—CH₂CHF₂), 2,2,2-trifluoroethyl (—CH₂CF₃), 1,1-difluoroethyl (—CF₂CH₃), 2-fluoropropyl (—CH₂CHFCH₃), 1,1-difluoropropyl (—CF₂CH₂CH₃), 2,2-difluoropropyl (—CH₂CF₂CH₃), 3,3-difluoropropyl (—CH₂CH₂CHF₂), 3,3,3-trifluoropropyl (—CH₂CH₂CHF₃), 1,1-difluorobutyl (—CF₂CH₂CH₂CH₃), perfluoroethyl (—CF₂CF₃), perfluoropropyl (—CF₂CF₂CF₃), 1,1,2,2,3,3-hexafluorobutyl (—CF₂—CF₂CF₂CH₃), perfluorobutyl (—CF₂CF₂CF₂CF₃), 1,1,1,3,3,3-hexafluoropropan-2-yl (—CH₂(CF₃)₂) groups, and the like. Additional groups wherein the halogen substitution is with bromine, iodine, or chlorine atoms are also understood for use herein.

The term “haloalkoxy” refers to a haloalkyl group bonded through an oxygen atom. Non-limiting examples include fluoroalkoxy groups such as fluoromethoxy, difluoromethoxy, trifluoromethoxy, perflouroethoxy, 2,2-difluoroethoxy, 1,1,2,2,3,3-hexafluorobutoxy, and 2,2,3,3,3-pentafluorobutoxy groups. Additional haloalkoxy groups include those wherein the halogen substitution is with bromine, iodine, or chlorine atoms are also understood for use herein.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula (I), and a guest (or co-former) molecule or molecules. In particular embodiments the pharmaceutically acceptable co-crystal of the compound of Formula (I) with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

The term “co-crystal” also means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

The term “pharmaceutically acceptable salt” or “therapeutically acceptable salt” refer to a salt form of a compound of Formula (I) which is, within the scope of sound medical evaluation, suitable for use in contact with the tissues and organs of humans and/or animals such that any resulting toxicity, irritation, allergic response, and the like and are commensurate with a reasonable benefit/risk ratio.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The terms “carrier” or “pharmaceutically acceptable carrier” refer to an excipient or vehicle that includes without limitation diluents, disintegrants, precipitation inhibitors, surfactants, glidants, binders, lubricants, and the like with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

### Results and Discussion

### The Aminoguanidine Robenidine is an Effective Antimalarial In Vitro

Robenidine is a symmetrical aminoguanidine drug originally developed for the treatment of Eimeria-derived coccidiosis in poultry and livestock.⁴ Recent investigations by Trott, McCluskey and others have demonstrated that the aminoguanidine chemotype can be modified and reapplied to pathogens other than Eimeria.³¹ Prior to the present study, robenidine had not been tested for antimalarial activity against P. falciparum despite the close genetic relationship between Plasmodium and Eimeria.

To gain an initial assessment of robenidine as a potential antimalarial drug, it was evaluated in vitro for the ability to inhibit the growth of P. falciparum strain D6, a drug-sensitive strain. Blood stage parasite cultures were incubated in a range of concentrations of robenidine, and 72 hour parasite growth was measured by SYBR green staining relative to untreated controls.³⁸ In this assessment, robenidine exhibited an average in vitro IC₅₀ of 324 nM (FIG. 2). This value represents an excellent starting point for further evaluation of structure activity relationships of robenidine analogs. Notably, the concentration-response curve was characterized by a steep slope at the IC₅₀ inflection point, similar to the control drug chloroquine.

### Synthesis of Aminoguanidine Robenidine Analogs

Given the successful initial evaluation of robenidine in vitro, a series of 38 robenidine analogs were designed and synthesized to determine the potential for improved antimalarial activity within the aminoguanidine chemotype. Robenidine analogs were synthesized in a single step route starting from commercially-available substituted benzaldehydes or acetophenones with the synthetic approach utilized in robenidine's initial discovery.⁴ Two equivalents of each starting material were refluxed in ethanol with 1,3-diaminoguanidine hydrochloride (Scheme 1). The resulting symmetrical aryl aminoguanidine products (formed as HCl salts) were crashed out of solution using diethyl ether, filtered, and washed with diethyl ether. Compounds were purified by recrystallization from ethanol.

As evidenced by the continued commercial success of robenidine as a veterinary drug, this synthetic approach is highly amenable to affordable, large scale synthesis, an important attribute given that any new antimalarial drug must be inexpensive for use in resource poor regions where the disease is endemic. It is noteworthy that the aminoguanidines can be prepared in a single step from commercially available starting materials, and no chromatographic separations are required for their purification. This is a significant advantage over other antimalarial drug candidates requiring multiple steps and/or complex separations.

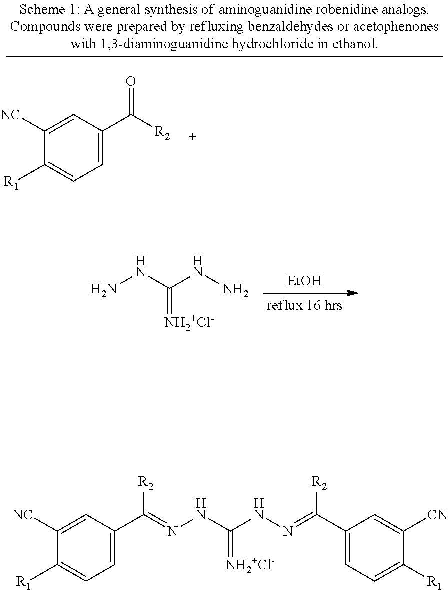

### Structure Activity Relationships of Aminoguanidines In Vitro

The in vitro activity of each compound in the aminoguanidine library was assessed against asynchronous cultures of Plasmodium falciparum parasites replicating within human erythrocytes (Tables 1-3). As the chemical development of robenidine was not discussed in its original publication in 1970⁴ and limited structural information is available regarding its mechanism of action,¹⁰ profiling was guided primarily by in vitro potency against the P. falciparum D6 strain cultured in human erythrocytes as described above.

Robenidine is structured as a diarylaminoguanidine with symmetrical 4-chloro (para relative to the aminoguanidine moiety) substituents on its two phenyl rings. Our SAR studies primarily focused on the potential to improve antimalarial activity by exchanging these 4-chloro substituents for different functional groups at the same position. All compounds are symmetrical and take the form shown in Scheme 1 unless otherwise noted. A halogen series (Table 1) was prepared to examine the effects of both halide type and position on antiplasmodial activity. Within this series, chloro and bromo substituted aminoguanidines were more active than fluoro substituted compounds, with position effects varying among the halides. The ortho-fluoro compound 2 displayed much lower activity than robenidine, and the para-bromo compound 9 showed moderately improved activity.

Other replacement functional groups for robenidine's 4-chloro substituents were varied widely for size, lipophilicity, and electron withdrawing vs. donating effects on the adjacent phenyl ring. Compounds with promising activity were also prepared as their ortho and meta isomers to investigate positional effects for these functional groups. Similarly, promising compounds were prepared with additional methyl groups on the benzyl carbons of the aminoguanidine moieties as shown in Scheme 1 (the R2 position) by starting from the analogous acetophenones rather than benzaldehydes.

The 4-methoxy robenidine derivative 11 was among the earliest compounds to show improved potency over robenidine. To further pursue this activity, the methyl (10), trifluoromethyl (12), trifluoromethoxy (16), and difluoromethoxy (18) derivatives were also prepared along with their R₂-methyl analogs (Table 2). Among these compounds, Compound 16 was the most potent, quickly becoming the frontrunner with a nearly ten-fold reduction in IC₅₀ value (39 nM vs. D6) relative to robenidine (324 nM vs. D6). Conversion of the R₂ moiety from H to methyl did not have a consistent effect, improving activity for the trifluoromethyl derivative (12 vs. 13) while reducing activities of the trifluoromethoxy and difluoromethoxy derivatives (16 vs 17 and 18 vs 19, respectively). The ortho (14) and meta (15) variants of Compound 16 were also prepared and demonstrated significantly reduced antimalarial activity in comparison with the para isomer.

The early success of Compound 16 led to an interest in exploring other electron withdrawing groups at the para position. The nitro (27) and cyano (22) derivatives were significantly more active than robenidine. 22 in particular had activity in the same range as the early hit compound 16, so the ortho (20) and meta (21) analogs were prepared to explore the position effects of the nitrile group. This series was expected to show a similar pattern to the trifluoromethoxy compounds, and so it came as quite a surprise when the 3-CN analog 21 was found to have IC₅₀ value of 7 nM, much lower than any other aminoguanidine evaluated up to that point. The R₂-methyl analog (23) of this compound was prepared and found to be less active than the R₂—H original.

A few general trends were observed for in vitro antimalarial activity. In general, substitution at the ortho position dramatically reduced antimalarial activity, possibly by sterically constraining rotation of the aryl rings. The carbonate 29 and tetrazoles 34 and 35 were inactive against all tested P. falciparum strains. Electron withdrawing substituents appear to have a positive effect on antimalarial activity, though the biphenyl analog 26 is unusually potent and the dimethylamino (28) and propynoxy (32) compounds also exhibit respectable antiparasitic activity.

All aminoguanidines synthesized were also evaluated against three multi-drug resistant P. falciparum strains (Dd2, Tm90-C2B, and A6) and for mammalian cell cytotoxicity. Robenidine and the lead compounds 16, 21, and 22 were further evaluated in vivo to guide optimization resulting in the design of Compound 1, the overall series lead. The results of these experiments are listed in Tables 1-3 and discussed below.

TABLE 1
Aminoguanidine halogen series and antimalarial drugs^{a}

| | | | | | IC₅₀ | | IC₅₀ | |
|---|---|---|---|---|---|---|---|---|
| | | | IC₅₀ D6 | IC₅₀ Dd2 | Tm90-C2B | IC₅₀ A6 | Cytotoxicity | |
| Compound | R₁ | R₂ | (nM) | (nM) | (nM) | (nM) | (μM) | IVTI |
| 2 | 2-F | H | 2866 | 4593 | >5000 | 1893 | >200 | >100 |
| 3 | 3-F | H | 767 | >5000 | >5000 | 426 | 49 | 64 |
| 4 | 4-F | H | 1016 | 3792 | 4904 | 369 | 24 | 24 |
| 5 | 2-Cl | H | 334 | 961 | 1219 | 348 | >200 | >100 |
| 6 | 3-Cl | H | 202 | 567 | 664 | 237 | 35 | >100 |
| Robenidine | 4-Cl | H | 324 | 814 | 1317 | 410 | 30 | 93 |
| 7 | 2-Br | H | 280 | 715 | 974 | 876 | 94 | >100 |
| 8 | 3-Br | H | 572 | 1516 | 1838 | 502 | 62 | >100 |
| 9 | 4-Br | H | 277 | 876 | 899 | 269 | 38 | >100 |
| Atovaquone | N/A | N/A | <1 | <1 | >5000 | >5000 | 72 | >100 |
| Chloroquine | N/A | N/A | 10 | 70 | 297 | 16 | 181 | >100 |

^{a}See Scheme for the aminoguanidine scaffold and sites of modification. P. falciparum IC₅₀ values are the average of two to four determinations, each carried out in quadruplicate (a more granular view of this data is provided in the supporting information). D6, P. falciparum pan-sensitive strain; Dd2, multi-drug resistant P. falciparum strain; Tm90-C2B, multi-drug resistant P. falciparum clinical isolate that is also resistant to atovaquone; A6, P. falciparum in-house derived mutant line resistant to respiratory antagonists.⁴⁰
Cytotoxicity assays were carried out with human hepatoma derived HepG2 cells and performed in quadruplicate.
IVTI = In Vitro Therapeutic Index, defined as cytotoxicity/D6 IC₅₀.
N/A = not applicable.

TABLE 2
Aminoguanidine methyl, methoxy, fluoromethyl, fluoromethoxy, and nitrile series^{a}

| | | | | | IC₅₀ | IC₅₀ | IC₅₀ | |
|---|---|---|---|---|---|---|---|---|
| | | | IC₅₀ D6 | IC₅₀ Dd2 | Tm90-C2B | A6 | Cytotoxicity | |
| Compound | R₁ | R₂ | (nM) | (nM) | (nM) | (nM) | (μM) | IVTI |
| 10 | 4-CH₃ | H | 905 | 2427 | 2529 | >5000 | 43 | 48 |
| 11 | 4-OCH₃ | H | 263 | 1602 | >5000 | 289 | >200 | >100 |
| 12 | 4-CF₃ | H | 614 | 1536 | 2742 | 567 | 40 | 65 |
| 13 | 4-CF₃ | CH₃ | 211 | 414 | 451 | 283 | 114 | >100 |
| 14 | 2-OCF₃ | H | 1080 | 2328 | >5000 | 391 | 156 | >100 |
| 15 | 3-OCF₃ | H | 670 | 1571 | 2988 | 689 | 61 | 91 |
| 16 | 4-OCF₃ | H | 39 | 83 | 114 | 52 | 11 | >100 |
| 17 | 4-OCF₃ | CH₃ | 140 | 526 | 458 | 182 | 9 | 64 |
| 18 | 4-OCHF₂ | H | 71 | 191 | 193 | 87 | 19 | >100 |
| 19 | 4-OCHF₂ | CH₃ | 99 | 403 | 315 | 171 | 3 | 30 |
| 20 | 2-CN | H | 1024 | 2097 | 1990 | 969 | 31 | 30 |
| 21 | 3-CN | H | 7 | 20 | 31 | 24 | 7 | >100 |
| 22 | 4-CN | H | 58 | 166 | 312 | 76 | >200 | >100 |
| 23 | 3-CN | CH₃ | 47 | 70 | 104 | 17 | 9 | >100 |
| 1 | 3-CN, 4-F | H | 4 | 12 | 16 | 14 | 8 | >100 |

^{a}See Table 1 legend and Experimental section.

TABLE 3
Other aminoguanidines^{a}

| | | | | | IC₅₀ | | IC₅₀ | |
|---|---|---|---|---|---|---|---|---|
| | | | IC₅₀ D6 | IC₅₀ Dd2 | Tm90-C2B | IC₅₀ A6 | Cytotoxicity | |
| Compound | R₁ | R₂ | (nM) | (nM) | (nM) | (nM) | (μM) | IVTI |
| 24 | H | H | 705 | 4298 | >5000 | 581 | >200 | >100 |
| 25 | 4-OH | H | 143 | 240 | 286 | 278 | 39 | >100 |
| 26 | 4-Ph | H | 15 | 87 | 71 | 33 | >200 | >100 |
| 27 | 4-NO₂ | H | 96 | 198 | 247 | 85 | >200 | >100 |
| 28 | 4-NMe₂ | H | 118 | 264 | 213 | 122 | 20 | >100 |
| 29 | 4-COOH | H | >5000 | >5000 | >5000 | >5000 | >200 | N/A |
| 30 | 4-CONH₂ | H | 527 | 405 | 727 | 516 | 42 | 80 |
| 31 | 4-SO₂NH₂ | H | >5000 | >5000 | >5000 | >5000 | >200 | N/A |
| 32 | 4-Propynoxy | H | 102 | 199 | 376 | 153 | >200 | >100 |
| 33 | 4-Morpholino | H | 131 | 329 | 295 | 116 | >200 | >100 |
| 34 | 3-Tetrazole | H | 1645 | >5000 | >5000 | 4790 | 107 | 65 |
| 35 | 4-Tetrazole | H | 838 | >5000 | >5000 | >5000 | >200 | N/A |
| 36 | (2,2-DiFluoro) | H | 250 | 800 | 905 | 235 | >200 | >100 |
| | 2,3-Dioxazole | | | | | | | |
| 37 | 2,4-OMe, 5-Cl | CH₃ | 360 | 1247 | 1180 | 216 | 3 | 8 |
| 38 | mono 2-F^{b} | H | 1028 | 2202 | >5000 | 833 | >200 | >100 |

^{a}See Table 1 legend and Experimental section.
^{b}Compound 38 has a 2-Fluoro substituent at only one R₁ site. The other R₁ site is unsubstituted (4-H).

### Aminoguanidines Retain In Vitro Activity in Drug-Resistant Strains of Malaria

In addition to the drug-sensitive P. falciparum D6 strain, the aminoguanidines were assessed against three drug-resistant strains (Tables 1-3). P. falciparum Dd2 is a multidrug resistant strain sensitive to atovaquone but resistant to chloroquine as well as the antifolate combination of pyrimethamine+sulfadoxine. P. falciparum Tm90-C2B is a multidrug resistant clinical isolate including resistance to both atovaquone and chloroquine.³⁹ P. falciparum A6 is derived from D6 and is resistant to respiratory antagonists such as atovaquone and antimycin A but sensitive to chloroquine.⁴⁰

The degree of cross resistance observed for the MDR strains Dd2 and C2B with the tested aminoguanidine series ranged from extensive (e.g., ˜19-fold for 11), to intermediate (e.g., 6.5-fold for 3), to modest (2-4-fold for 1 and 23) relative to the drug sensitive D6 strain of P. falciparum. The general lack of significant cross-resistance is consistent with expectations given that robenidine and other aminoguanidines are not clinically prescribed for malaria or even administered directly to humans for any indication (it is possible that trace amounts of robenidine have passed into humans via the consumption of poultry treated for coccidiosis, though these trace amounts are unlikely to drive antimalarial resistance).

Aminoguanidines have High In Vitro Therapeutic Indices

The aminoguanidines were evaluated for cytotoxicity against human hepatoma derived HepG2 cells (Tables 1-3). In this assay, HepG2 cells were incubated with test compounds for 24 hours, followed by a 24-hour recovery period and subsequent staining to evaluate for cytotoxic effects with resazurin. The ratio of the resulting HepG2 IC₅₀ value to the P. falciparum D6 IC₅₀ value can be considered an ‘in vitro therapeutic index,’ or IVTI.

Cytotoxicity in Hep2G did not track proportionally with antimalarial activity in any of the four tested P. falciparum strains. Overall, substitution at the 3 (meta) position resulted in increased cytotoxicity relative to the 2 (ortho) and 4 (para) positions, though the cyano substituent was shown to be an exception to this trend. Several of the aminoguanidines, including the active compound 21, had no measurable effect on Hep2G activity at concentrations as high as 200 μM. Most aminoguanidines in the series had HepG2 IC₅₀ values above 10 μM, and nearly all active aminoguanidines had an in vitro therapeutic index of over 1000-fold (this value cannot be calculated for those aminoguanidines having no antimalarial activity). It is noteworthy that 1, with the greatest antiplasmodial activity among compounds in this series, exhibits an IVTI value of 2,000 which is indicative of its highly selective antiparasitic action.

### Aminoguanidine Activity is Concentration Dependent but not Time Dependent

Many drugs have activity dependent on their exposure time in addition to concentration, and antimalarial potency is frequently stage-specific. To determine whether the aminoguanidines acted by a time-dependent mechanism against malaria parasites, the SYBR Green activity assay was adapted to include additional incubation intervals. Two potent aminoguanidines, 16 and 21, were incubated with P. falciparum Dd2 infected erythrocytes for 48, 72, or 96 hours (FIG. 3). Notably, this assay measures drug-treated parasite growth during the incubation time relative to untreated parasites. A shorter incubation time allows for less parasite growth for all drug conditions, producing data with proportionally greater variability or ‘noise.’ Conversely, longer incubation times reduce noise in the resulting data.

IC₅₀ values for both 16 and 21 were somewhat higher at 48 hours than at longer drug incubation times (Table 4), though this may be the result of noise in the data associated with the short incubation time. This finding may also stem from the use of asynchronous parasite cultures, wherein one life-cycle stage may be more impacted than another (16 in particular appears to exhibit a biphasic concentration-response curve, indicative of stage specificity). IC₅₀ values for both compounds were virtually identical between 72-hour and 96-hour incubation time points. These results indicate that a 72-hour incubation may be required to attain full in vitro activity, but that no additional benefit is construed with longer incubation times. Aminoguanidine activity appears to be driven primarily by drug concentration rather than by lengthening drug incubation time. Additional experiments are planned to explore for possible stage-specific activity of the most active compounds in this series however our results combine to suggest that the molecules are not acting in a manner consistent with a “delayed death” phenotype as shown for other drugs such as doxycycline and azithromycin.⁴⁴

TABLE 4
Pf Dd2 IC₅₀ of 16 and 21 vs drug incubation length^{a}

| | IC₅₀ Dd2 (nM) | | |
|---|---|---|---|
| Compound | 48 hr | 72 hr | 96 hr |
| 16 | 410 | 195 | 192 |
| 21 | 31 | 21 | 22 |

^{a}See Table 1 legend and Experimental section.

Lead Aminoguanidines have High In Vivo Efficacy in a Mouse Model of Malaria

Aminoguanidines exhibiting high in vitro antimalarial potency and robenidine were assessed for in vivo efficacy in a murine model of malaria (Plasmodium yoelii, Table 5). In this modified 4-day Peters test,⁴¹ mice were inoculated with parasites from a donor mouse (day 0), and then dosed orally with drugs on each of the subsequent four days (days 1-4). On day 5 of the experiment, the parasitemia for each mouse was determined microscopically by examining methanol-fixed and Giemsa-stained blood smears. The ED₅₀ represents the interpolated dose of a compound at which parasitemia was suppressed to one half that of untreated controls. Similarly, the ED₉₀ represents the dose at which parasitemia is suppressed ten-fold. Mice were considered cured of their infection if no parasites were detected in the blood 30 days from the first drug administration, and the non-recrudescence dose (NRD) represents the lowest dose to achieve a cure.

Robenidine, 16, 22, and 21 were evaluated for in vivo antimalarial efficacy. That robenidine exhibited respectable in vivo antimalarial activity was somewhat surprising given that it is poorly absorbed and known to accumulate in the gastrointestinal tract. The early hit compound 16 (the 4-OCF₃ analog, ED₅₀ 1.2 mg/kg/day) showed improved in vivo activity over robenidine (ED₅₀=1.6 mg/kg/day), while 22 (the 4-CN analog, ED₅₀=2.7 mg/kg/day) did not. Unexpectedly, 21 (the 3-CN analog, ED₅₀=7.1 mg/kg/day) was six-fold less efficacious than 16 in vivo, despite being six-fold more active than 16 in vitro. For comparative purposes consider that the ED₅₀ of chloroquine in this system is 1.5 mg/kg/day.⁴²

The only aminoguanidine which produced a cure in this model was 16 with NRD of 12.5 mg/kg/day, while other compounds were not curative (including compound 1 described below). It is important to note that failure to produce a cure in this model and by this dosing regimen is not predictive of clinical failure. Indeed, several approved clinical drugs such as chloroquine are not curative in this model at any dose level.

TABLE 5
in vivo ED₅₀ and ED₉₀ of lead aminoguanidines^{a}

| | P. yoelii ED₅₀ | P. yoelii ED₉₀ | P. yoelii NRD |
|---|---|---|---|
| Compound | (mg/kg/day) | (mg/kg/day) | (mg/kg/day) |
| Robenidine | 1.6 | 4.9 | >25 |
| 16 | 1.2 | 3.7 | 12.5 |
| 22 | 2.7 | 7.7 | >25 |
| 21 | 7.1 | 9.4 | >25 |
| 1 | 0.25 | 0.28 | >25 |

^{a}In vivo activity values were determined from a modified 4-day Peters test.
Compounds were administered by oral gavage up to 25 mg · kg/day, near the solubility limit of the PEG delivery vehicle.
NRD = non-recrudescence dose (cure dose).

### Compound 1 is a Highly Potent Antimalarial In Vitro and In Vivo

The discrepancy between the in vitro success (Table 2) and in vivo mediocrity (Table 5) of 21 remained a mystery which we later explored. From previous in vivo SAR studies on other scaffolds, we had noted that aryl groups without protective substitutions at the para positions were biologically unstable. While 16 was substituted at the para position, 21 was not, potentially leaving this position vulnerable to hepatic microsomal degradation.

To interrogate this hypothesis, an analog of 21 was prepared with an additional para-fluoro substituent (1, 3-CN, 4-F). Given the low activity of 4 (4-F), this substitution was expected to have little effect on in vitro potency while potentially improving upon the in vivo activity of 21. Unexpectedly, 1 had excellent in vitro activity, becoming the new series lead in potency. 1 had a single-digit nanomolar IC₅₀ value against D6 (4 nM), IC₅₀ values in the low double digits for the drug resistant strains, and an in vitro therapeutic index of 2000-fold.

The in vivo efficacy of Compound 1 was even more pronounced with an ED₅₀ value of 0.25 mg/kg/day, fivefold lower than its nearest competitor 16. The single atom difference between 21 (3-CN, 4-H) and 1 (3-CN, 4-F) resulted in a nearly 30-fold improvement in in vivo efficacy.

Murine Microsomal Stability of Aminoguanidines Correlates with In Vivo Activity

To investigate the relationship between the aminoguanidines' in vivo efficacy and their metabolic properties, the murine microsomal stability of a selection of aminoguanidines was evaluated (Table 6). Robenidine, 16, 21, 1, and the control compound ketanserin were incubated with pooled murine liver microsomes and monitored for degradation by LC/MS/MS for one hour. The concentration vs. time plot for each compound was used to determine its biological half-life (t_{1/2}) and predicted intrinsic clearance (Clᵢₙₜ).

For all of the aminoguanidines evaluated, murine microsomal stability correlated with in vivo efficacy. Robenidine, 16, and 1 were all biologically stable with half-lives above 150 min (with the same rank order for in vivo activity and stability). 21 was metabolically unstable in the presence of murine microsomes, with a half-life of only 58.18 min.

This data supports the notion that 1 is more efficacious in vivo than 21 due in part to improved stability. Substituting the 4-position H for F resulted in a three-fold increase in metabolic stability. Presumably the prolonged presence of Compound 1 in the bloodstream contributes to its excellent performance in vivo.

TABLE 6
Murine Microsomal Stability of Aminoguanidines^{a}

| | Murine Microsomal | Predicted Clᵢₙₜ |
|---|---|---|
| Compound | Stability, t^{1/2} (min) | (mL/min/kg) |
| Ketanserin | 14.47 | 377.20 |
| Robenidine | 158.65 | 34.40 |
| 16 | 172.16 | 31.70 |
| 21 | 58.18 | 93.80 |
| 1 | 186.15 | 29.32 |

^{a}Data from ChemPartner Co. Ltd, Shanghai, P.R. China.
See Experimental section for full details.

### Conclusions

Compound 1 is a robenidine derivative with excellent in vitro potency, virtually no cross-resistance in multi-drug resistant strains, and a high in vitro therapeutic index. In a murine model of malaria, 1 displayed robust in vivo antimalarial activity propelled by a combination of high intrinsic potency and biological stability. Although speculative at this time it also possible that the nitrogen atoms in the aminoguanidine bridge of Compound 1 exhibit diminished basicity (i.e., ionizeabilty) due to the presence of two strongly electron withdrawing groups (F and CN) at the para and meta positions of the flanking aromatic rings which may in turn enhance oral bioavailability.

Further exploration of the aminoguanidine scaffold is certainly warranted, as is developing more knowledge of its antimalarial properties including the mode of action. Assessing the in vivo activity of these compounds in humanized mice may refine predictions of clinical success. Assessing the compounds against synchronous parasites will elucidate potential stage-specific potency effects. Beyond the blood stage, evaluating the aminoguanidines against other stages of the malaria life cycle such as the liver stage will provide valuable information useful for their potential development for use in humans. Systematically exploring the mechanism of action including chemical biology techniques and resistance studies can further guide SAR for this chemotype, and we are currently engaged in this work. Potential synergy with other antimalarial compounds such as artemisinin, atovaquone, and ELQ-300 will also be assessed.

As we look for new entries in the antimalarial pipeline, it may be useful to reexamine drugs and chemotypes effective in related parasites and pathogens. This appears to be the case with robenidine, a drug discovered in 1970 but which has not been methodically explored in malaria using a medicinal chemistry approach until this point. Though robenidine itself has reasonably good antimalarial activity in vitro and in vivo, it did not take long to improve upon this activity in both settings. In this study, a second look at an old drug efficiently produced a new and promising chemical lead.

### Methods

### General Chemistry—Materials and Instruments

All solvents, starting materials, and reagents were acquired from commercial sources (Sigma-Aldrich and Combi-Blocks). Robenidine was obtained from Santa Cruz Biotechnology (Santa Cruz, Calif.). All materials were used without further purification. ¹H and ¹³C NMR Spectra were taken on a Bruker 400 MHz instrument and chemical shifts are reported relative to TMS (0.0 ppm). Fluorescence measurements were recorded using a Molecular Devices Spectramax iD3 equipped with Softmax Pro 7 software. Final compounds were measured to be >95% pure by high performance liquid chromatography (HPLC) using an Agilent Technologies 1260 Infinity II system (unless otherwise noted). High-resolution mass spectrometry (HRMS) using electrospray ionization was performed by the Portland State University BioAnalytical Mass Spectrometry Facility. Melting points were measured using a Stanford Research Systems OptiMelt Automated Melting Point System (model MPA100).

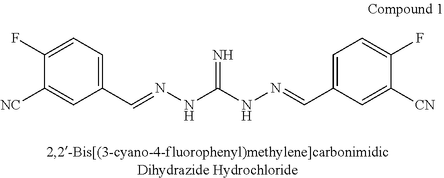 [SMILES: N#Cc1cc(/C=N/NC(=N)N/N=C/c2ccc(F)c(C#N)c2)ccc1F]

A solution of 3-cyano-4-fluorobenzaldehyde (1.31 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.54 g, 99%. ¹H NMR (400 MHz, DMSO-d6) δ 12.56 (s, 2H), 8.76 (s, 2H), 8.64 (d, J=5.63 Hz, 2H), 8.46 (s, 2H), 8.33-8.32 (m, 2H), 7.68 (t, J=8.92, 2H). ¹³C NMR (400 MHz, DMSO-d6) δ 164.98, 162.40, 153.57, 146.25, 136.94, 135.95, 133.22, 131.61, 131.57, 117.73, 117.53, 114.22, 101.56, 101.40. HRMS found 352.1111, M+H. MP=317-319° C.

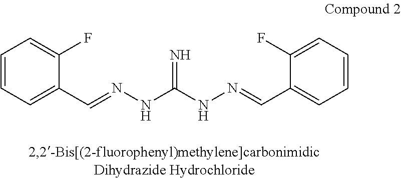 [SMILES: N=C(N/N=C/c1ccccc1F)N/N=C/c1ccccc1F]

A solution of 2-fluorobenzaldehyde (1.1 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.34 g, 99%. ¹H NMR (400 MHz, DMSO-d6) δ 12.54 (s, 2H), 8.72 (s, 2H), 8.64 (s, 2H), 8.36 (dt, J=7.9, 1.72, 2H), 7.59-7.53 (m, 2H), 7.34 (t, J=7.9 Hz, 4H). ¹³C NMR (400 MHz, DMSO-d6) δ 162.21, 159.71, 152.74, 141.62, 132.93, 127.31, 124.75, 120.82, 116.07, 115.86. HRMS found 302.1206, M+H. MP=293-295° C.

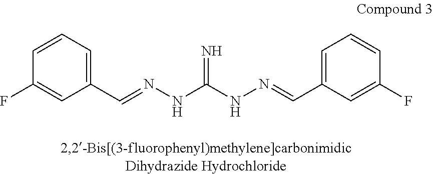 [SMILES: N=C(N/N=C/c1cccc(F)c1)N/N=C/c1cccc(F)c1]

A solution of 3-fluorobenzaldehyde (1.09 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.31 g, 97%. ¹H NMR (400 MHz, DMSO-d6) δ 12.56 (s, 2H), 8.69 (s, 2H), 8.48 (s, 2H), 7.98 (d, J=10.3 Hz, 2H), 7.70 (d, 7.6 Hz, 2H), 7.53 (q, J=7.3, 2H), 7.33 (t, J=8.3, 2H). ¹³C NMR (400 MHz, DMSO-d6) δ 164.14, 161.72, 153.47, 148.02, 136.37, 136.28, 131.34, 131.26, 125.37, 118.15, 117.94, 113.89, 113.66. HRMS found 302.1206, M+H. MP=281-283° C.

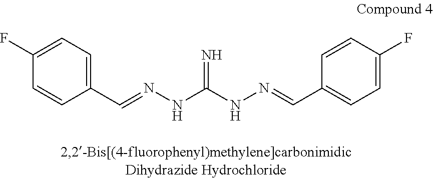 [SMILES: N=C(N/N=C/c1ccc(F)cc1)N/N=C/c1ccc(F)cc1]

A solution of 4-fluorobenzaldehyde (1.09 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.33 g, 99%. ¹H NMR (400 MHz, DMSO-d6) δ 12.32 (s, 2H), 8.56 (s, 2H), 8.44 (s, 2H), 8.03 (t, J=6.5 Hz, 4H), 7.34 (t, J=8.2, 4H). ¹³C NMR (400 MHz, DMSO-d6) δ 165.24, 162.77, 153.41, 148.14, 130.71, 130.62, 130.48, 130.45, 116.40, 116.19. HRMS found 302.1205, M+H. MP=295-297° C.

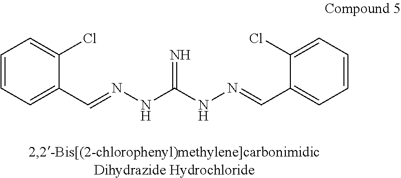 [SMILES: N=C(N/N=C/c1ccccc1Cl)N/N=C/c1ccccc1Cl]

A solution of 2-chlorobenzaldehyde (1.23 g, 8.8 mmol, 2.2 eq.) and 1,3-diaminoguanidine hydrochloride (500 g, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.42 g, 96%. ¹H NMR (400 MHz, DMSO-d6) δ 12.62 (s, 2H), 8.88 (s, 2H), 8.69 (s, 2H), 8.43 (d, J=7.15 Hz, 2H), 7.58-7.46 (m, 6H). ¹³C NMR (400 MHz, DMSO-d6) δ 153.19, 145.53, 134.06, 132.77, 131.03, 130.39, 128.51, 127.95. HRMS found 334.0620, M+H. MP=295-297° C.

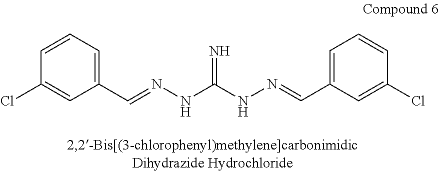 [SMILES: N=C(N/N=C/c1cccc(Cl)c1)N/N=C/c1cccc(Cl)c1]

A solution of 3-chlorobenzaldehyde (1.23 g, 8.8 mmol, 2.2 eq.) and 1,3-diaminoguanidine hydrochloride (500 g, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.48 g, 100%. ¹H NMR (400 MHz, DMSO-d6) δ 12.39 (s, 2H), 8.69 (s, 2H), 8.43 (s, 2H), 8.17 (s, 2H), 7.83 (d, J=6.49 Hz, 2H), 7.59-7.50 (m, 2H). ¹³C NMR (400 MHz, DMSO-d6) δ 153.40, 147.93, 135.91, 134.27, 131.11, 130.92, 127.71, 127.06. HRMS found 334.0619, M+H. MP=268-270° C.

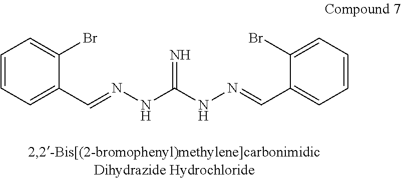 [SMILES: N=C(N/N=C/c1ccccc1Br)N/N=C/c1ccccc1Br]

A solution of 2-bromobenzaldehyde (1.23 g, 8.8 mmol, 2.2 eq.) and 1,3-diaminoguanidine hydrochloride (500 g, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.83 g, 100%. ¹H NMR (400 MHz, DMSO-d6) δ 12.52 (s, 2H), 8.80 (s, 2H), 8.66 (s, 2H), 8.39 (d, J=7.64 Hz, 2H), 7.73 (d, J=7.94, 2H), 7.51 (t, J=7.43 Hz, 2H), 7.43 (t, J=7.56, 2H). ¹³C NMR (400 MHz, DMSO-d6) δ 153.21, 133.66, 133.00, 132.53, 128.87, 128.44, 124.44. HRMS found 423.9586, M+H. MP=279-281° C.

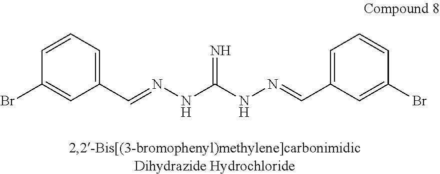 [SMILES: N=C(N/N=C/c1cccc(Br)c1)N/N=C/c1cccc(Br)c1]

A solution of 3-bromobenzaldehyde (1.63 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.80 g, 98%. ¹H NMR (400 MHz, DMSO-d6) δ 12.28 (s, 2H), 8.66 (s, 2H), 8.39 (s, 2H), 8.29 (t, J=3.08 Hz, 2H), 7.87 (d, J=7.80, 2H), 7.68 (dd, J=8.00, 2.79, 2H), 7.45 (t, J=7.86, 2H). ¹³C NMR (400 MHz, DMSO-d6) δ 136.13, 133.80, 131.34, 129.95, 128.05, 122.83. HRMS found 423.9589, M+H. MP=258-260° C.

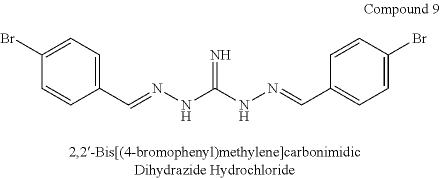 [SMILES: N=C(N/N=C/c1ccc(Br)cc1)N/N=C/c1ccc(Br)cc1]

A solution of 4-bromobenzaldehyde (1.63 g, 8.8 mmol, 2.2 eq.) and 1,3-diaminoguanidine hydrochloride (500 g, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.73 g, 94%. ¹H NMR (400 MHz, DMSO-d6) δ 12.32 (s, 2H), 8.61 (s, 2H), 8.41 (s, 2H), 7.91 (d, J=8.24 Hz, 4H), 7.70 (d, J=8.21, 4H). ¹³C NMR (400 MHz, DMSO-d6) δ 153.32, 148.29, 133.05, 132.23, 130.23, 124.70. HRMS found 423.9587, M+H. MP=295-297° C.

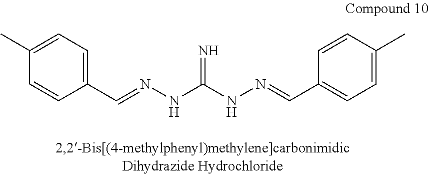

A solution of 4-methylbenzaldehyde (1.06 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.11 g, 84%. ¹H NMR (400 MHz, DMSO-d6) δ 12.18 (s, 2H), 8.46 (s, 2H), 8.39 (s, 2H), 7.83 (d, J=7.8 Hz, 4H), 7.30 (d, J=7.8 Hz, 4H), 2.37 (s, 6H). ¹³C NMR (400 MHz, DMSO-d6) δ 152.08, 148.22, 140.13, 130.00, 128.74, 127.24, 20.51. HRMS found 294.1708, M+H. MP=246-248° C.

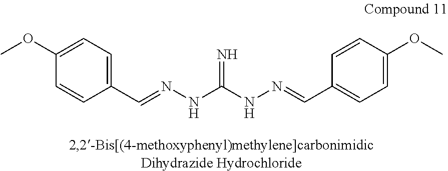

A solution of 4-methoxybenzaldehyde (1.2 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.37 g, 94%. ¹H NMR (400 MHz, DMSO-d6) δ 12.00 (s, 2H), 8.37 (s, 2H), 8.35 (s, 2H), 7.88 (d, J=9.1 Hz, 4H), 7.04 (d, J=9.1 Hz, 4H), 3.83 (s, 6H). ¹³C NMR (400 MHz, DMSO-d6) δ 181.34, 152.55, 129.56, 125.90, 114.22, 55.38. HRMS found 326.1608, M+H. MP=218-220° C.

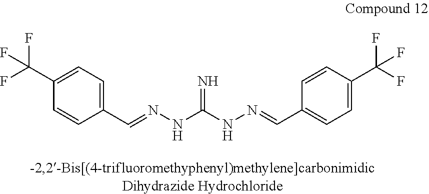 [SMILES: N=C(N/N=C/c1ccc(C(F)(F)F)cc1)N/N=C/c1ccc(C(F)(F)F)cc1]

A solution of trifluoromethylbenzaldehyde (1.53 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.75 g, 100%. ¹H NMR (400 MHz, DMSO-d6) δ 12.55 (s, 2H), 8.78 (s, 2H), 8.55 (s, 2H), 8.19 (d, J=7.8 Hz, 4H), 7.85 (d, J=8.1, 4H). ¹³C NMR (400 MHz, DMSO-d6) δ 153.12, 147.44, 137.21, 130.44, 130.12, 128.51, 125.53, 125.39, 122.68. HRMS found 402.1140, M+H. MP=273-275° C.

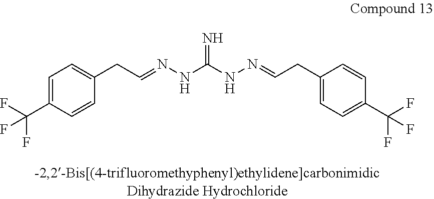 [SMILES: N=C(N/N=C/Cc1ccc(C(F)(F)F)cc1)N/N=C/Cc1ccc(C(F)(F)F)cc1]

A solution of 4-trifluoromethylacetophenone (1.65 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.78 g, 96%. ¹H NMR (400 MHz, DMSO-d6) δ 12.04 (s, 2H), 8.89 (s, 2H), 8.28 (d, J=7.6 Hz, 4H), 7.81 (d, J=8.4, 4H), 2.5 (s, 6H). ¹³C NMR (400 MHz, DMSO-d6) δ 154.75, 141.00, 128.26, 125.63, 123.28. HRMS found 430.1455, M+H. MP=334-336° C.

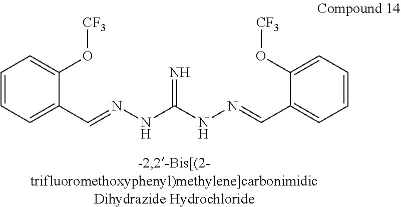 [SMILES: N=C(N/N=C/c1ccccc1OC(F)(F)F)N/N=C/c1ccccc1OC(F)(F)F]

A solution of 2-trifluoromethoxybenzaldehyde (1.67 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.87 g, 100%. ¹H NMR (400 MHz, DMSO-d6) δ 12.54 (s, 2H), 8.75 (s, 2H), 8.71 (s, 2H), 8.48 (d, J=7.8 Hz, 2H), 7.67-7.50 (m, 6H). ¹³C NMR (400 MHz, DMSO-d6) δ 152.21, 146.36, 131.98, 127.28, 127.14, 125.67, 121.03, 120.74, 118.19. HRMS found 434.1039, M+H. MP=194-196° C.

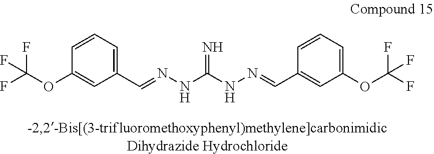 [SMILES: N=C(N/N=C/c1cccc(OC(F)(F)F)c1)N/N=C/c1cccc(OC(F)(F)F)c1]

A solution of 3-trifluoromethoxybenzaldehyde (835 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 660 mg, 70%. ¹H NMR (400 MHz, DMSO-d6) δ 12.43, (s, 2H), 8.71 (s, 2H), 8.47 (s, 2H), 8.09 (s, 2H), 7.93 (d, J=7.6 Hz, 2H), 7.63 (t, J=7.9, 2H), 7.49 (d, J=8.3 Hz, 2H). ¹³C NMR (400 MHz, DMSO-d6) δ 153.48, 149.29, 147.93, 136.22, 131.31, 128.06, 123.45, 121.84, 120.13. HRMS found 434.1039, M+H. MP=241-243° C.

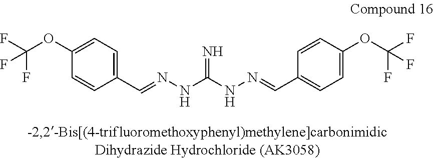 [SMILES: N=C(N/N=C/c1ccc(OC(F)(F)F)cc1)N/N=C/c1ccc(OC(F)(F)F)cc1]

A solution of 4-trifluoromethoxybenzaldehyde (1.67 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.03 g, 55%. ¹H NMR (400 MHz, DMSO-d6) δ 12.24 (s, 2H), 8.61 (s, 2H), 8.45 (s, 2H), 8.10 (d, J=8.9 Hz, 4H), 7.50 (d, J=8.1 Hz, 4H). ¹³C NMR (400 MHz, DMSO-d6) δ 150.26, 133.07, 130.34, 121.70. HRMS found 434.1041, M+H. MP=278-280° C.

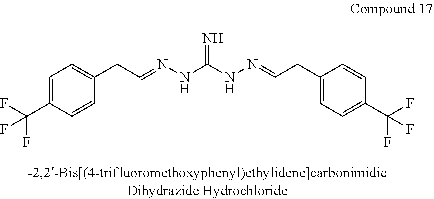 [SMILES: N=C(N/N=C/Cc1ccc(C(F)(F)F)cc1)N/N=C/Cc1ccc(C(F)(F)F)cc1]

A solution of 4-trifluoromethoxyacetophenone (1.79 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.88 g, 95%. ¹H NMR (400 MHz, DMSO-d6) δ 11.91 (s, 2H), 8.78 (s, 2H), 8.19 (d, J=8.8 Hz, 4H), 7.44 (d, J=8.1, 4H), 2.46 (s, 6H). ¹³C NMR (400 MHz, DMSO-d6) δ 154.66, 149.85, 136.38, 129.60, 121.79, 121.17, 119.24. HRMS found 462.1352, M+H. MP=323-325° C.

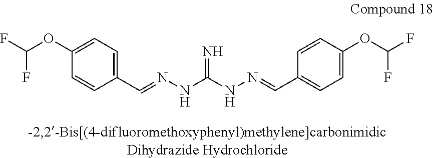 [SMILES: N=C(N/N=C/c1ccc(OC(F)F)cc1)N/N=C/c1ccc(OC(F)F)cc1]

A solution of 4-difluoromethoxybenzaldehyde (757 mg, 4.4 mmol, 2.2 eq.) and 1,3-diaminoguanidine hydrochloride (250 g, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 0.63 g, 73%. ¹H NMR (400 MHz, DMSO-d6) δ 12.28 (s, 2H), 8.56 (s, 2H), 8.43 (s, 2H), 8.02 (d, J=8.6, 4H), 7.38 (t, J=74, 2H), 7.29 (d, J=8.6 Hz, 4H). ¹³C NMR (400 MHz, DMSO-d6) δ 153.17, 130.67, 130.22, 119.01, 116.58. HRMS found 398.1231, M+H. MP=245-247° C.

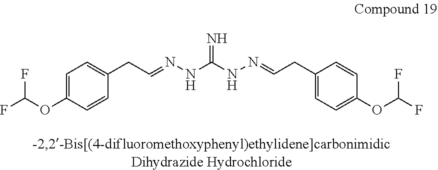 [SMILES: N=C(N/N=C/Cc1ccc(OC(F)F)cc1)N/N=C/Cc1ccc(OC(F)F)cc1]

A solution of 4-difluoromethoxyacetophenone (818 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 990 mg, 100%. ¹H NMR (400 MHz, DMSO-d6) δ 11.78 (s, 2H), 8.72 (s, 2H), 8.13 (d, J=8.8 Hz, 4H), 7.36 (t, J=73.56, 2H), 7.24 (d, J=8.6, 2H). ¹³C NMR (400 MHz, DMSO-d6) δ 154.53, 152.90, 152.66, 133.97, 129.39, 119.19, 118.54, 116.62, 114.06, 15.33. HRMS found 426.1538, M+H. MP=273-275° C.

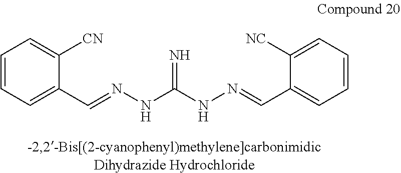 [SMILES: N#Cc1ccccc1/C=N/NC(=N)N/N=C/c1ccccc1C#N]

A solution of 2-cyanobenzaldehyde (1.15 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.23 g, 88%. ¹H NMR (400 MHz, DMSO-d6) δ 12.88 (s, 2H), 8.8 (s, 2H), 8.74 (s, 2H), 8.52 (d, J=7.8 Hz, 2H), 7.96 (d, J=7.8 Hz, 2H), 7.84 (t, J=7.7 Hz, 2H), 7.68 (t, J=7.7, 2H), 7.34 (t, J=50.59, 2H). ¹³C NMR (400 MHz, DMSO-d6) δ 145.01, 136.32, 133.91, 133.88, 131.56, 127.55, 117.67, 111.51, 56.48, 19.03. HRMS found 316.1301, M+H. MP=213-215° C.

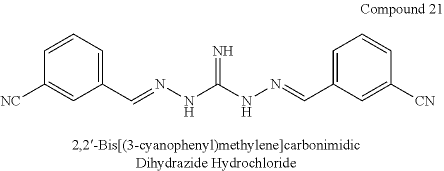 [SMILES: N#Cc1cccc(/C=N/NC(=N)N/N=C/c2cccc(C#N)c2)c1]

A solution of 3-cyanobenzaldehyde (1.15 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.40 g, 100%. ¹H NMR (400 MHz, DMSO-d6) δ 12.44 (s, 2H), 8.74 (s, 2H), 8.56 (s, 2H), 8.46 (s, 2H), 8.22 (d, J=8.1 Hz, 2H), 7.95 (d, J=7.8 Hz, 2H), 7.71 (t, J=7.6 Hz, 2H). ¹³C NMR (400 MHz, DMSO-d6) δ 153.60, 147.27, 135.10, 134.21, 133.25, 131.24, 130.50, 118.95, 112.49. HRMS found 316.1300, M+H. MP=278-280° C.

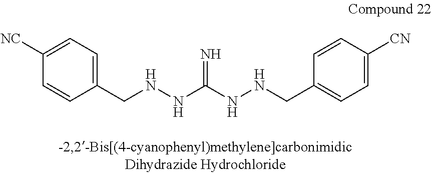 [SMILES: N#Cc1ccc(CNNC(=N)NNCc2ccc(C#N)cc2)cc1]

A solution of 4-cyanobenzaldehyde (1.15 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.40 g, 100%. ¹H NMR (400 MHz, DMSO-d6) δ 12.69 (s, 2H), 8.79 (s, 2H), 8.52 (s, 2H), 8.17 (d, J=8.2 Hz, 4H), 7.97 (d, J=8.3 Hz, 4H). ¹³C NMR (400 MHz, DMSO-d6) δ 153.64, 147.70, 138.14, 133.11, 128.92, 119.09, 113.05. HRMS found 316.1303, M+H. MP=303-305° C.

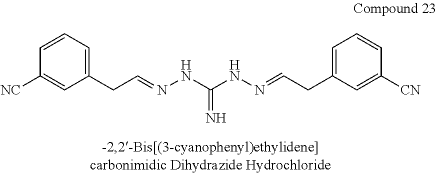 [SMILES: N#Cc1cccc(C/C=N/NC(=N)N/N=C/Cc2cccc(C#N)c2)c1]

A solution of 3-cyanoacetophenone (1.28 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.40 g, 92%. ¹H NMR (400 MHz, DMSO-d6) δ 11.89 (s, 2H), 8.90 (s, 2H), 8.63 (t, J=1.44 Hz, 2H), 8.33 (dt, J=7.75, 1.39, 2H), 7.92 (dt, J=7.76, 2.44, 2H), 7.67 (t, J=7.88, 2H), 2.48 (s, 6H). ¹³C NMR (400 MHz, DMSO-d6) δ 154.70, 152.16, 138.21, 133.59, 132.06, 131.04, 130.09, 119.21, 112.16, 15.29. HRMS found 344.1612, M+H. MP=304-306° C.

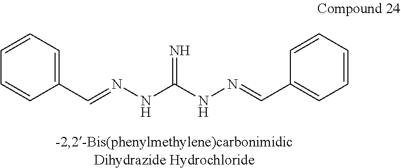 [SMILES: N=C(N/N=C/c1ccccc1)N/N=C/c1ccccc1]

A solution of benzaldehyde (933 mg, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.19 g, 99%. ¹H NMR (400 MHz, DMSO-d6) δ 12.23 (s, 2H), 8.53 (s, 2H), 8.44 (s, 2H), 7.96-7.94 (m, 4H), 7.50-7.49 (m, 6H). ¹³C NMR (400 MHz, DMSO-d6) δ 133.27, 130.77, 128.73, 127.86. HRMS found 266.1398, M+H. MP=245-247° C.

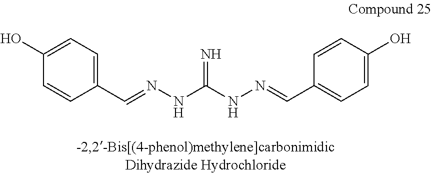 [SMILES: N=C(N/N=C/c1ccc(O)cc1)N/N=C/c1ccc(O)cc1]

A solution of 4-hydroxybenzaldehyde (1.1 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.33 g, 100%. ¹H NMR (400 MHz, DMSO-d6) δ 11.95 (s, 2H), 10.15 (s, 2H), 8.28 (s, 2H), 7.75 (d, J=8.1 Hz, 4H), 6.87 (d, J=8.3, 4H). ¹³C NMR (400 MHz, DMSO-d6) δ 160.56, 130.15, 124.76, 116.10. HRMS found 298.1296, M+H. MP=185-187° C.

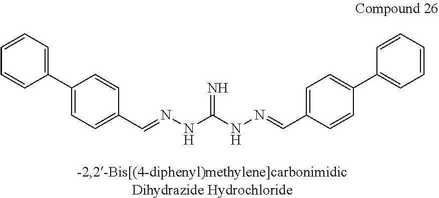 [SMILES: N=C(N/N=C/c1ccc(-c2ccccc2)cc1)N/N=C/c1ccc(-c2ccccc2)cc1]

A solution of 4-phenylbenzaldehyde (1.60 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.81 g, 100%. ¹H NMR (400 MHz, DMSO-d6) δ 12.32 (s, 2H), 8.59 (s, 2H), 8.49 (s, 2H), 8.05 (d, J=7.3 Hz, 2H), 7.81 (d, J=7.3 Hz, 2H), 7.77 (d, J=7.6 Hz, 2H), 7.51 (t, J=7.3 Hz, 2H), 7.42 (t, J=7.6 Hz, 1H). ¹³C NMR (400 MHz, DMSO-d6) δ 153.28, 142.71, 139.72, 132.89, 129.52, 128.99, 128.51, 127.42, 127.28. HRMS found 418.2022, M+H. MP=286-288° C.

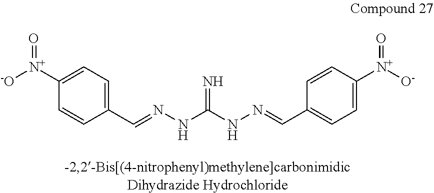

A solution of 4-nitrobenzaldehyde (1.33 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.56 g, 100%. ¹H NMR (400 MHz, DMSO-d6) δ 12.68 (s, 2H), 8.82 (s, 2H), 8.57 (s, 2H), 8.33 (d, J=8.3 Hz, 4H), 8.24 (d, J=8.3 Hz, 4H). ¹³C NMR (400 MHz, DMSO-d6) δ 153.79, 148.78, 147.27, 139.98, 129.35, 124.35. HRMS found 356.1098, M+H. MP=285-287° C.

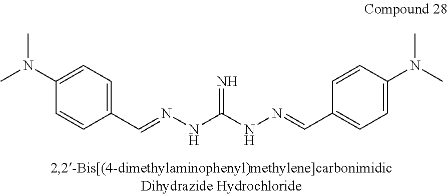

A solution of N, N-dimethylaminobenzaldehyde (1.31 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 90 mg, 6%. This compound was chemically unstable in chromatography solvents, but was greater than 80% pure when used in in vitro assays. ¹H NMR (400 MHz, DMSO-d6) δ 11.74 (s, 2H), 8.22 (s, 2H), 8.14 (s, 2H), 7.71 (d, J=7.8 Hz, 4H), 6.75 (d, J=7.8 Hz, 4H), 3.00 (s, 6H). ¹³C NMR (400 MHz, DMSO-d6) δ 152.50, 152.35, 129.69, 129.41, 121.22, 120.96, 112.01. HRMS found 352.2237, M+H. MP=132-134° C.

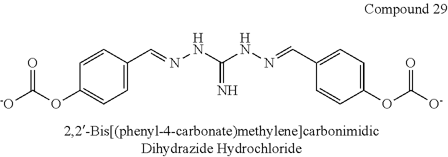

A solution of 4-formylphenylcarbonate (1.32 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 600 mg, 39%. ¹H NMR (400 MHz, DMSO-d6) δ 13.13 (s, 2H), 12.57 (s, 2H), 8.70 (s, 2H), 8.52 (s, 2H), 8.11 (d, J=7.7 Hz, 4H), 8.03 (d, J=7.7 Hz, 4H). ¹³C NMR (400 MHz, DMSO-d6) δ 193.48, 167.31, 167.02, 153.47, 148.46, 139.35, 137.72, 136.12, 132.79, 130.39, 130.03, 128.40. HRMS found 354.1195, M+H. MP=336-338° C.

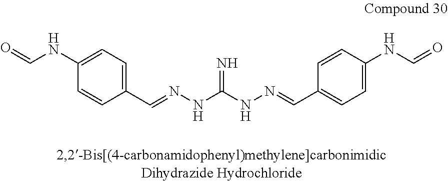 [SMILES: N=C(N/N=C/c1ccc(NC=O)cc1)N/N=C/c1ccc(NC=O)cc1]

A solution of 4-carbonamidobenzaldehyde (655 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 770 mg, 100%. ¹H NMR (400 MHz, DMSO-d6) δ 12.43 (s, 2H), 8.85 (s, 2H), 8.50 (s, 2H), 8.13 (s, 2H), 8.04 (d, J=7.8 Hz, 4H), 7.98 (d, J=7.8 Hz, 4H), 7.50 (s, 2H). ¹³C NMR (400 MHz, DMSO-d6) δ 167.70, 153.41, 148.59, 136.32, 136.26, 128.30, 128.15. HRMS found 352.5100, M+H. MP=309-311° C.

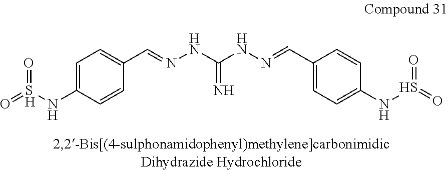

A solution of 4-sulphonamidobenzaldehyde (814 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 920 mg, 100%. ¹H NMR (400 MHz, DMSO-d6) δ 12.41, (s, 2H), 8.69 (s, 2H), 8.50 (s, 2H), 8.14 (6, J=7.6 Hz, 4H), 7.91 (d, J=7.6, 4H), 7.49 (s, 4H). ¹³C NMR (400 MHz, DMSO-d6) δ 153.49, 146.00, 136.81, 128.72, 125.43. HRMS found 424.0852, M+H. MP=285-287° C.

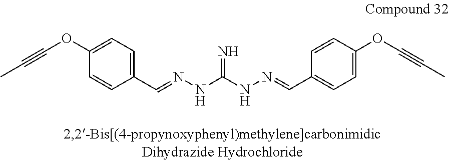 [SMILES: CC#COc1ccc(/C=N/NC(=N)N/N=C/c2ccc(OC#CC)cc2)cc1]

A solution of 4-propynoxybenzaldehyde (704 mg, 4.4 mmol, 2.2 eq.) and 1,3-diaminoguanidine hydrochloride (250 g, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 0.82 g, 100%. ¹H NMR (400 MHz, DMSO-d6) δ 12.19 (s, 2H), 8.42 (s, 2H), 8.37 (s, 2H), 7.91 (d, J=8.4 Hz, 4H). ¹³C NMR (400 MHz, DMSO-d6) δ 159.67, 153.09, 129.93, 127.06, 115.56, 79.38, 79.06, 56.09. HRMS found 374.1606, M+H. MP=217-219° C.

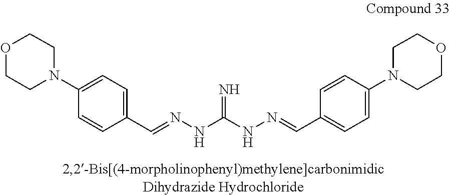 [SMILES: N=C(N/N=C/c1ccc(N2CCOCC2)cc1)N/N=C/c1ccc(N2CCOCC2)cc1]

A solution of 4-morpholinobenzaldehyde (840 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 850 mg, 90%. ¹H NMR (400 MHz, DMSO-d6) δ 11.84 (s, 2H), 8.26 (s, 4H), 7.77 (d, J=8.1 Hz, 4H), 7.01 (d, J=8.1 Hz, 4H), 3.75 (s, 8H), 3.24 (s, 8H). ¹³C NMR (400 MHz, DMSO-d6) δ 153.09, 152.75, 149.18, 129.58, 123.87, 114.5366.40, 47.78. HRMS found 436.2449, M+H. MP=280-282° C.

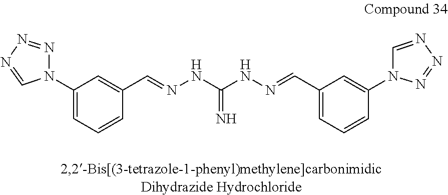 [SMILES: N=C(N/N=C/c1cccc(-n2cnnn2)c1)N/N=C/c1cccc(-n2cnnn2)c1]

A solution of 3-tetrazole-1-benzaldehyde (766 mg, 4.4 mmol, 2.2 eq.) and 1,3-diaminoguanidine hydrochloride (250 g, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 870 mg, 100%. This compound was not sufficiently soluble in chromatography solvents to obtain a quantitative purity by HPLC. ¹H NMR (400 MHz, DMSO-d6) δ 8.84 (s, 2H), 8.75 (s, 2H), 8.66 (s, 2H), 8.20 (t, J=9.0 Hz, 4H), 7.76 (t, J=7.78, 2H). ¹³C NMR (400 MHz, DMSO-d6) δ 152.27, 147.44, 133.85, 129.83, 129.27, 128.30, 125.70, 124.25. HRMS found 402.1641, M+H. MP=289-291° C.

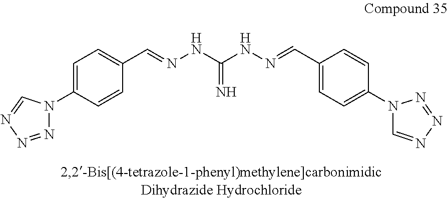 [SMILES: N=C(N/N=C/c1ccc(-n2cnnn2)cc1)N/N=C/c1ccc(-n2cnnn2)cc1]

A solution of 4-tetrazole-1-benzaldehyde (1.53 g, 8.8 mmol, 2.2 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 1.75 g, 100%. This compound was not sufficiently soluble in chromatography solvents to obtain a quantitative purity by HPLC. ¹H NMR (400 MHz, DMSO-d6) δ 8.70 (s, 2H), 8.52 (s, 2H), 8.20 (s, 8H), 3.47-3.42 (m, 2H), 1.06 (t, J=7.0 Hz, 2H). ¹³C NMR (400 MHz, DMSO-d6) δ 153.39, 148.45, 136.23, 129.19, 127.77. HRMS found 402.1641, M+H. MP=277-279° C.

### Compound 36-2,2′-Bis{[4-(2,2-difluoro-2,3-dioxazolo)]phenyl}methylene]carbonimidic Dihydrazide Hydrochloride

A solution of 4-(2,2-difluoro-2,3-dioxazolo)benzaldehyde (820 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 800 mg, 87%. ¹H NMR (400 MHz, DMSO-d6) δ 12.58 (s, 2H), 8.66 (s, 2H), 8.60 (s, 2H), 8.04 (d, J=7.74 Hz, 2H), 7.52 (dd, J=7.97, 0.93, 2H), 7.32 (t, J=8.12, 2H). ¹³C NMR (400 MHz, DMSO-d6) δ 142.55, 140.90, 140.27, 130.60, 123.91, 120.71, 116.23, 111.11. HRMS found 426.0814, M+H. MP=276-278° C.

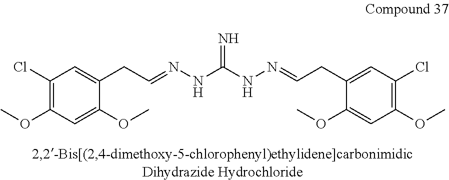

A solution of 2,4-dimethoxy-5-chloroacetophenone (655 mg, 4.4 mmol, 2.2 eq.) and 1,3-diaminoguanidine hydrochloride (250 mg, 2 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. Yield: 400 mg, 39%. ¹H NMR (400 MHz, DMSO-d6) δ 11.64 (s, 2H), 8.55 (s, 2H), 7.68 (s, 2H), 6.85 (s, 2H), 3.94 (s, 6H), 3.91 (s, 6H), 2.31 (s, 6H). ¹³C NMR (400 MHz, DMSO-d6) δ 157.97, 156.84, 154.64, 154.53, 130.97, 130.65, 120.76, 112.75, 98.42, 98.30, 57.21, 56.96, 56.79, 32.07, 19.31. HRMS found 482.1350, M+H. MP=229-231° C.

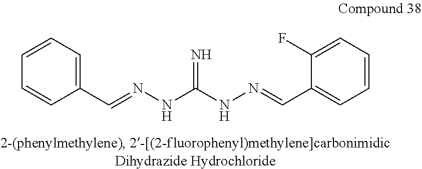 [SMILES: N=C(N/N=C/c1ccccc1)N/N=C/c1ccccc1F]

A solution of benzaldehyde (466 mg, 4.4 mmol, 1.1 eq.), 2-fluorobenzaldehyde (546 mg, 4.4 mmol, 1.1 eq.) and 1,3-diaminoguanidine hydrochloride (500 mg, 4 mmol, 1 eq.) in ethanol (5 mL) was refluxed for 16 hr. Diethyl ether (10 mL) was added and the product carbonimidic dihydrazide crashed out of solution as a white solid. The product was filtered, washed with diethyl ether, and recrystallized from methanol as a hydrochloride salt. This product was isolated as a mixture also containing 24 and 2. 38 was the dominant product accounting for greater than 50% of the total material in the mixture. Yield: 1.27 g mixture, 50%. ¹H NMR (400 MHz, DMSO-d6) δ 12.30 (s, 3H), 8.69 (s, 1H), 8.62 (s, 1H), 8.57 (s, 1H), 8.53 (s, 1H), 8.44 (s, 1H), 8.35 (t, J=6.5 Hz, 1H), 7.96-7.94 (m, 2H), 7.56 (q, J=8.1 Hz, 1H), 7.51-7.49 (m, 2H), 7.34 (t, J=8.1, 2H). ¹³C NMR (400 MHz, DMSO-d6) δ 133.27, 130.78, 128.74, 127.89, 127.28, 124.80, 115.88. HRMS found 284.1301, M+H. MP=281-283° C.

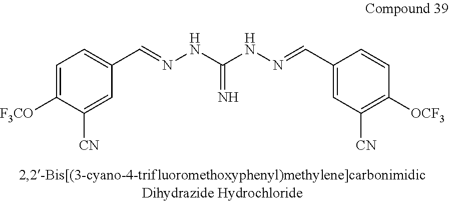 [SMILES: N#Cc1cc(/C=N/NC(=N)N/N=C/c2ccc(OC(F)(F)F)c(C#N)c2)ccc1OC(F)(F)F]

A solution of 3-cyano-4-trifluoromethoxybenzaldehyde (95 mg, 0.44 mmol, 2.2 eq.) and 1,3-diaminoguanidine hydrochloride (25 mg, 0.2 mmol, 1 eq.) in ethanol (5 mL) was refluxed for 16 hr. Diethyl ether (5 mL) was added and the product carbonimidic dihydrazide crashed out of solution as a white solid. The product was filtered, washed with diethyl ether, and recrystallized from methanol as a hydrochloride salt. Yield: 45 mg, 43%. MP=292-294° C.

### General Biology—Parasite Culture and Drug Sensitivity

The following parasite strains were used in this study and obtained through BEI Resources, NIAID, NIH. Plasmodium falciparum, Strain D6 (MRA-285, originally from Sierra Leone, has modest resistance to mefloquine).⁴⁵ Strain Dd2 (MRA-150, originated from Indochina; derived from W2-mef and is resistant to chloroquine, pyrimethamine and mefloquine. P. falciparum strain Tm90-C2B (Thailand; resistant to mefloquine, chloroquine, atovaquone, and pyrimethamine) was obtained directly from the Division of Experimental Therapeutics of Walter Reed Army Institute of Research (WRAIR) in Silver Spring, Md., USA.³⁹ Strain SB1-A6 (MRA-1002, Sierra Leone was derived from D6 clone and is resistant to Atovaquone and ELQ-300.⁴⁰ P. falciparum parasites were thawed from frozen stock and cultured in suspended human erythrocytes (Lampire Biological Labs, Pipersville, Pa.) not more than 28 days old at 2% hematocrit. The culture medium used was RPMI-1640, supplemented with 25 mM HEPES buffer, 25 mg/L gentamicin sulfate, 45 mg/L hypoxanthine, 10 mM glucose, 2 mM glutamine, and 0.5% Albumax II (complete medium).⁴³ Cultures were maintained in a standard low oxygen atmosphere (5% O₂, 5% CO₂, 90% N₂) in an environmental chamber and incubated at 37° C. Cultures were sub-passaged every 3-4 days into a fresh culture flask containing complete media and erythrocytes.

### IC₅₀ Determination by the Fluorescence-Based SYBR Green Assay

The aminoguanidine series was assessed for in vitro antiplasmodial activity using the fluorescence-based SYBR Green assay described previously by Smilkstein and co-workers.³⁸ Compounds were evaluated in quadruplicate in flat-bottomed Costar clear 96-well plates (Model #3585). A two-fold serial dilution of each compound was performed across the columns of the test plates starting with 20 μM and ending with a final untreated column to serve as control wells. Asynchronous parasite infected erythrocytes in growth media were added to each well for a total volume of 100 μL, final hematocrit of 2%, and initial parasitemia of 0.2%. The commercial malaria drugs atovaquone and chloroquine were used as control drugs. Test plates were incubated for 72 hr at 37° C. in an environmental chamber with a controlled low oxygen atmosphere (5% O₂, 5% CO₂, 90% N₂). After the incubation period, 100 μL SYBR Green I dye-detergent solution was added to each well, and the plates were incubated at ambient temperature and atmosphere in the dark for at least one hour. Fluorescence was read at 497 nm excitation and 520 nm emission bands using a Spectramax iD3 plate reader. Fluorescence readings were normalized with respect to the untreated control wells representing normal parasite growth and plotted against the logarithm of drug concentration. An IC₅₀ was determined for each compound by fitting this data to a variable slope nonlinear regression curve using Graphpad Prism software (v. 8).

### HepG2 Cytotoxicity Assay

Compounds were prepared as 10 mM stock solutions in DMSO. Human hepatocarcinoma (HepG2) cells were maintained in culture at 37° C. in a humidified 5% CO₂ atmosphere in RPMI-1640 medium containing 10% fetal bovine serum. HepG2 cells were added to each well of flat bottom 96-well tissue culture plates at an initial density of 2×10⁴ cells per well, and an initial volume of 160 μL complete medium per well. After an overnight incubation at 37° C. to adhere the cells to the culture plates, 40 μL drug solutions in complete medium were applied to each well at a final concentration range of 0 to 200 μM across each plate. Drugs were tested in triplicate or quadruplicate. The cells were incubated for 24 hours at 37° C. and 5% CO₂ with the drug solutions, which were then aspirated and replaced with 200 μL per well of complete medium for an additional 24 hour incubation under the same conditions. To each well was added 20 μL of resazurin (Alamar Blue) in PBS buffer to a final concentration of 10 μM, and the plates were incubated for 3 hours. Fluorescence was measured at 560 nm excitation and 590 nm emission bands using a Spectramax iD3 plate reader. Fluorescence readings were normalized with respect to the untreated control wells and plotted against the logarithm of drug concentration. An IC₅₀ was determined for each compound by fitting this data to a variable slope nonlinear regression curve using Graphpad Prism software (v. 8).

### In Vivo Efficacy Against Murine Malaria

The in vivo ED₅₀ and ED₉₀ of selected aminoguanidines was measured using a modified 4-day Peters test. Female CF1 mice from Charles River Laboratories were inoculated intravenously with approximately 2.5-5.0×10⁴ parasitized erythrocytes (murine malaria P. yoelii, Kenya strain MR4 MRA-428) from a donor mouse (experiment day zero). On the following four days (experiment days 1-4), solutions of the test compounds in PEG-300 (or PEG-300 only for control mice) were administered by oral gavage once daily. Aminoguanidines were initially assessed at 2.5, 5, and 10 mg/kg/day, and experiments were repeated to adjust the dose range as needed to obtain an interpolated ED₅₀ and ED₉₀ value (1 required a dosing down to 0.1 mg/kg/day to attain this result). Experiments were repeated with doses up to 25 mg/kg/day to obtain a non-recrudescence dose, though only 16 was found to produce a cure in this model. Parasitemia of each mouse was determined by microscopic examination of Giemsa stained blood smears on day 5. ED₅₀ and ED₉₀ values were assessed by generating dose-response curves relative to untreated controls using Graphpad Prism (v. 8). Mice were considered cured of malarial infection if they maintained 0% parasitemia at experiment day 30. The procedures involved, together with all matters relating to the care, handling, and housing of the animals used in this study, were approved by the Portland VA Medical Center Institutional Animal Care and Use Committee.

### Murine Microsomal Stability

Metabolic stability studies of selected aminoguanidines were performed at ChemPartner, Shanghai, China. Compounds were incubated at 37° C. and 1 μM concentration in murine liver microsomes (Corning) for one hour at a protein concentration of 0.5 mg/mL in potassium phosphate buffer at pH 7.4 containing 1.0 mM EDTA. The metabolic reaction was initiated by NADPH and quenched with ice-cold acetonitrile at 15 minute increments up to one hour. The progress of compound metabolism was followed by LC-MS/MS (ESI positive ion, LC-MS/MS-034(API-6500+)) using a C18 stationary phase (ACQUITY UPLC BEH C18(2.1×50 mm, 1.7 μm) and a MeOH/water mobile phase containing 0.25% FA and 1 mM NH₄OAc. Imipramine or Osalmid were used as internal standards, and ketanserin was used as a metabolically unstable control compound. Concentration versus time data for each compound were fitted to an exponential decay function to determine the first-order rate constant for substrate depletion, which was then used to calculate the degradation half-life (t_{1/2}) and predicted intrinsic clearance value (Clᵢₙₜ) from an assumed murine hepatic blood flow of 90 mL/min/kg.

### REFERENCES

1. World Malaria Report 2020. Geneva: World Health Organization: 2020.
2. Dondorp et al., Artemisinin resistance in Plasmodium falciparum malaria. N Engl J Med 2009, 361, 455-467.
3. Hooft van Huijsduijnen et al., The antimalarial pipeline. Curr Opin Pharmacol 2018, 42, 1-6.
4. Kantor et al., 1,3-Bis(p-chlorobenzylideneamino)guanidine hydrochloride (robenzidene): new poultry anticoccidial agent. Science 1970, 168, 373-374.
5. Reid et al., Efficacy evaluations of robenzidene for control of coccidiosis in chickens. Avian Dis 1970, 14, 788-796.
6. Ryley et al., Studies on the mode of action of the coccidiostat robenidene. Z Parasitenkd 1971, 37, 85-93.
7. Ryley et al., The development of Eimeria brunetti in tissue culture. J Parasitol 1972, 58, 660-663.
8. Ryley et al., Comparative studies with anticoccidials and three species of chicken coccidia in vivo and in vitro. J Parasitol 1972, 58, 664-668.
9. Kennett et al., Efficacy studies with robenidine, a new type of anticoccidial, in the diet. Poult Sci 1974, 53, 978-986.
10. Wong et al., Robenzidene, an inhibitor of oxidative phosphorylation. Biochem Biophys Res Commun 1972, 46, 621-627.
11. Berger et al., Safety evaluation of robenidine in the feed of broiler chickens. Poult Sci 1974, 53, 1013-1015.
12. Long et al., Studies on Eimeria grenieri in the guinea fowl (Numida meleagris). Parasitology 1978, 76, 1-9.
13. Peeters et al., Efficacy of robenidine in the prevention of rabbit coccidiosis. Br Vet J 1979, 135, 349-354.
14. Wilson et al., Complete gene map of the plastid-like DNA of the malaria parasite Plasmodium falciparum. J Mol Biol 1996, 261, 155-172.
15. Ito et al., Effects of robenidine and some other drugs on mice experimentally infected with toxoplasma. Natl Inst Anim Health Q (Tokyo) 1974, 14, 129-136.
16. Krylov et al., [Khimotsid for treatment and prevention of toxoplasmosis]. Veterinariia 1976, 11, 69-71.
17. Bakhtari et al., Chemotherapy of experimental toxoplasmosis with special reference to robenidine. Folia Parasitol (Praha) 1988, 35, 193-198.
18. Ricketts et al., Toxoplasma gondii: susceptibility and development of resistance to anticoccidial drugs in vitro. Antimicrob Agents Chemother 1993, 37, 2358-2363.
19. Mukhopadhyay et al., Antileishmanial effect of a potent S-adenosylmethionine decarboxylase inhibitor: CGP 40215A. Pharmacol Res 1996, 33, 67-70.
20. Yao et al., Inhibitory effects of 19 antiprotozoal drugs and antibiotics on Babesia microti infection in BALB/c mice. J Infect Dev Ctries 2015, 9, 1004-1010.
21. Huang et al., Babesia microti Aldo-keto Reductase-Like Protein Involved in Antioxidant and Anti-parasite Response. Front Microbiol 2017, 8, 2006.
22. Bacchi et al., In vivo trypanocidal activities of new S-adenosylmethionine decarboxylase inhibitors. Antimicrob Agents Chemother 1996, 40, 1448-1453.
23. Brun et al., The story of CGP 40 215: studies on its efficacy and pharmacokinetics in African green monkey infected with Trypanosoma brucei rhodesiense. Trop Med Int Health 2001, 6, 362-368.
24. Beswick et al., Mechanisms of allosteric regulation of Trypanosoma cruzi S-adenosylmethionine decarboxylase. Biochemistry 2006, 45, 7797-7807.
25. Dec et al., Susceptibility of chicken Lactobacillus bacteria to coccidiostats. J Vet Med Sci 2020, 82, 333-336.
26. Stanley et al., Sensitivity of Escherichia coli O157:H7 strain 932 to selected anticoccidial drugs in broiler chicks. Poult Sci 1996, 75, 42-46.
27. Ogbunude et al., In vitro effect of diamidines on intracellular polyamines of Acanthamoeba polyphaga. Drugs Exp Clin Res 2001, 27, 127-133.
28. Molnar et al., Efficacy of some anticoccidial drugs for treating coccidial enteritis of the common carp caused by Goussia carpelli (Apicomplexa: Eimeriidae). Acta Vet Hung 2007, 55, 67-76.
29. Hansen et al., Fate and antibacterial potency of anticoccidial drugs and their main abiotic degradation products. Environ Pollut 2009, 157, 474-480.
30. Claes et al., The antibiotic robenidine exhibits guanabenz-like cytoprotective properties by a mechanism independent of protein phosphatase PP1:PPP1R15A. J Biol Chem 2019, 294, 13478-13486.
31. Abraham et al., Robenidine Analogues as Gram-Positive Antibacterial Agents. J Med Chem 2016, 59, 2126-2138.
32. Ogunniyi et al., Evaluation of robenidine analog NCL195 as a novel broad-spectrum antibacterial agent. PLoS One 2017, 12, e0183457.
33. Russell et al., Gram-Positive and Gram-Negative Antibiotic Activity of Asymmetric and Monomeric Robenidine Analogues. ChemMedChem 2018, 13, 2573-2580.
34. Russell et al., Discovery of 4,6-bis(2-((E)-benzylidene)hydrazinyl)pyrimidin-2-Amine with Antibiotic Activity. ChemistryOpen 2019, 8, 896-907.
35. Abraham et al., Aminoguanidines: New leads for treatment of Giardia duodenalis infection. Int J Parasitol Drugs Drug Resist 2019, 10, 38-44.
36. Khazandi et al., In vitro Antimicrobial Activity of Robenidine, Ethylenediaminetetraacetic Acid and Polymyxin B Nonapeptide Against Important Human and Veterinary Pathogens. Front Microbiol 2019, 10, 837.
37. Das Gupta et al., 3-Aminooxy-1-aminopropane and derivatives have an antiproliferative effect on cultured Plasmodium falciparum by decreasing intracellular polyamine concentrations. Antimicrob Agents Chemother 2005, 49, 2857-2864.
38. Smilkstein et al., Simple and inexpensive fluorescence-based technique for high-throughput antimalarial drug screening. Antimicrob Agents Chemother 2004, 48, 1803-1806.
39. Nilsen et al., Quinolone-3-diarylethers: a new class of antimalarial drug. Science translational medicine, 2013, 5, 177ra37.
40. Smilkstein et al., A drug-selected Plasmodium falciparum lacking the need for conventional electron transport. Mol Biochem Parasitol 2008, 159, 64-68.
41. Peters et al., The chemotherapy of rodent malaria. LX. The importance of formulation in evaluating the blood schizontocidal activity of some endoperoxide antimalarials. Ann Trop Med Parasitol 2002, 96, 559-573.
42. Pou et al., Sontochin as a guide to the development of drugs against chloroquine-resistant malaria. Antimicrob Agents Chemother 2012, 56, 3475-3480.
43. Trager et al., Human malaria parasites in continuous culture. Science 1976, 193, 673-675.
44. Dahl et al., Multiple antibiotics exert delayed effects against the Plasmodium falciparum apicoplast. Antimicrob Agents Chemother. 2007, 51, 485-490.
45. Oduola et al., Plasmodium falciparum: cloning by single-erythrocyte micromanipulation and heterogeneity in vitro. Exp. Parasitol. 1988, 66, 86-95

What is claimed:

1. A compound of Formula (I): 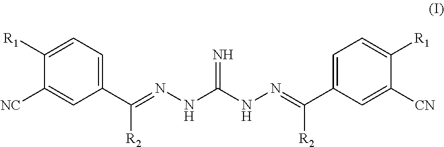
wherein:
R₁ is selected from the group of —F, —Cl, —Br, C₁-C₆ haloalkyl, C₁-C₆ haloalkoxy, —NO₂, —C(H)═O, ═O, —CN, and COOR₃;
R₂ is selected from the group of H and C₁-C₃ alkyl; and
R₃ is selected from the group of H, C₁-C₆ alkyl, and benzyl;
or a pharmaceutically acceptable salt, co-crystal, ester, solvate, hydrate, isomer, tautomer, isotope, polymorph, or pharmaceutically acceptable prodrug thereof.

2. The compound of claim 1, wherein:
R₁ is selected from the group of —F, —Cl, —Br, C₁-C₃ haloalkyl, and C₁-C₃ haloalkoxy;
R₂ is selected from the group of H and C₁-C₃ alkyl; and
or a pharmaceutically acceptable salt, co-crystal, ester, solvate, hydrate, isomer, tautomer, isotope, polymorph, or pharmaceutically acceptable prodrug thereof.

3. The compound of claim 1, wherein:
R₁ is selected from the group of —F, C₁-C₃ haloalkyl, and C₁-C₃ haloalkoxy;
R₂ is selected from the group of H and C₁-C₃ alkyl; and
or a pharmaceutically acceptable salt, co-crystal, ester, solvate, hydrate, isomer, tautomer, isotope, polymorph, or pharmaceutically acceptable prodrug thereof.

4. The compound of claim 1, wherein:
R₁ is selected from the group of —F, —Cl, C₁-C₃ fluoroalkyl, and C₁-C₃ fluoroalkoxy;
R₂ is selected from the group of H and C₁-C₃ alkyl; and
or a pharmaceutically acceptable salt, co-crystal, ester, solvate, hydrate, isomer, tautomer, isotope, polymorph, or pharmaceutically acceptable prodrug thereof.

5. The compound of claim 1, wherein:
R₁ is selected from the group of —F, —Cl, C₁-C₂ fluoroalkyl, and C₁-C₂ fluoroalkoxy;
R₂ is selected from the group of H and C₁-C₃ alkyl; and
or a pharmaceutically acceptable salt, co-crystal, ester, solvate, hydrate, isomer, tautomer, isotope, polymorph, or pharmaceutically acceptable prodrug thereof.

6. The compound of claim 1, wherein:
R₁ is selected from the group of —F, —Cl, —CH₂F, CHF₂, —CF₃, —OCH₂F, —OCHF₂, and —OCF₃;
R₂ is selected from the group of H and C₁-C₃ alkyl; and
or a pharmaceutically acceptable salt, co-crystal, ester, solvate, hydrate, isomer, tautomer, isotope, polymorph, or pharmaceutically acceptable prodrug thereof.

7. The compound of claim 1, wherein:
R₁ is selected from the group of —F, —Cl, —CH₂F, CHF₂, —CF₃, —OCH₂F, —OCHF₂, and —OCF₃;
R₂ is selected from the group of H and —CH₃; and
or a pharmaceutically acceptable salt, co-crystal, ester, solvate, hydrate, isomer, tautomer, isotope, polymorph, or pharmaceutically acceptable prodrug thereof.

8. The compound of claim 1, wherein:
R₁ is selected from the group of —F, —Cl, —CF₃, and —OCF₃;
or a pharmaceutically acceptable salt, co-crystal, ester, solvate, hydrate, isomer, tautomer, isotope, polymorph, or pharmaceutically acceptable prodrug thereof.

9. The compound of claim 1, wherein:
R₁ is selected from the group of —F, OCF₃ and —Cl;
or a pharmaceutically acceptable salt, co-crystal, ester, solvate, hydrate, isomer, tautomer, isotope, polymorph, or pharmaceutically acceptable prodrug thereof.

10. The compound of claim 1, wherein R₂ is H; or a pharmaceutically acceptable salt, co-crystal, ester, solvate, hydrate, isomer, tautomer, isotope, polymorph, or pharmaceutically acceptable prodrug thereof.

11. The compound of claim 1, wherein R₂ is —CH₃; or a pharmaceutically acceptable salt, co-crystal, ester, solvate, hydrate, isomer, tautomer, isotope, polymorph, or pharmaceutically acceptable prodrug thereof.

12. The compound of claim 1, wherein the compound has the structure: 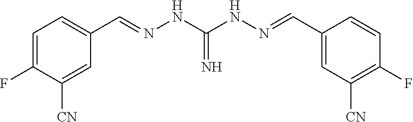 [SMILES: N#Cc1cc(/C=N/NC(=N)N/N=C/c2ccc(F)c(C#N)c2)ccc1F]
or a pharmaceutically acceptable salt, co-crystal, ester, solvate, hydrate, isomer, tautomer, isotope, polymorph, or pharmaceutically acceptable prodrug thereof.

13. The compound of claim 1, wherein the compound has the structure: 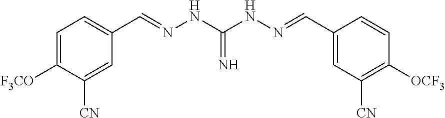 [SMILES: N#Cc1cc(/C=N/NC(=N)N/N=C/c2ccc(OC(F)(F)F)c(C#N)c2)ccc1OC(F)(F)F]
or a pharmaceutically acceptable salt, co-crystal, ester, solvate, hydrate, isomer, tautomer, isotope, polymorph, or pharmaceutically acceptable prodrug thereof.

14. A pharmaceutical composition comprising a pharmaceutically effective amount of a compound of claim 1, or a pharmaceutically acceptable salt, co-crystal, ester, solvate, hydrate, isomer, tautomer, isotope, polymorph, or pharmaceutically acceptable prodrug thereof, and a pharmaceutically acceptable carrier.

15. A method of treatment of a malaria infection in a subject, the method comprising administering to a subject in need thereof a pharmaceutically effective amount of a compound of claim 1, or a pharmaceutically acceptable salt, co-crystal, ester, solvate, hydrate, isomer, tautomer, isotope, polymorph, or pharmaceutically acceptable prodrug thereof.

16. The method of claim 15, wherein the malaria infection in the subject is caused by a Plasmodium species selected from the group of Plasmodium falciparum, Plasmodium vivax, Plasmodium ovale, Plasmodium malariae, and Plasmodium knowlesi.

17. The method of claim 16, wherein the Plasmodium species is resistant to one or more anti-malarial agents or combinations thereof, including those selected from the group of chloroquine, amodiaquine, atovaquone, sulphadoxine, pyrimethamine, mefloquine, sulphadoxine-pyrimethamine, quinine, piperaquine-mefloquine, mefloquine-artesunate, artemether-lumefantrine, dihydroaremisinin, artesunate, artmether, arteether, DHA-piperaquine and DHA-piperaquine mefloquine-artesunate; or a pharmaceutically acceptable salt thereof.

18. The method of claim 17, wherein the Plasmodium species is resistant to atovaquone, or a pharmaceutically acceptable salt thereof.

19. The method of claim 15, wherein the compound of claim 1 has the structure: 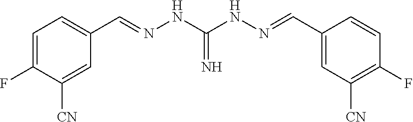 [SMILES: N#Cc1cc(/C=N/NC(=N)N/N=C/c2ccc(F)c(C#N)c2)ccc1F]
or a pharmaceutically acceptable salt, co-crystal, ester, solvate, hydrate, isomer, tautomer, isotope, polymorph, or pharmaceutically acceptable prodrug thereof.

20. The method of claim 15, wherein the compound of claim 1 has the structure: 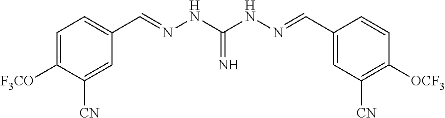 [SMILES: N#Cc1cc(/C=N/NC(=N)N/N=C/c2ccc(OC(F)(F)F)c(C#N)c2)ccc1OC(F)(F)F]
or a pharmaceutically acceptable salt, co-crystal, ester, solvate, hydrate, isomer, tautomer, isotope, polymorph, or pharmaceutically acceptable prodrug thereof.
